SEQUENCE LISTING (25 sequences)

SEQ ID NO: 1 - protein, 5 aa, synthetic construct
NYVMS

SEQ ID NO: 2 - protein, 17 aa, synthetic construct
TISSGGRYIYYTDSVKG

SEQ ID NO: 3 - protein, 10 aa, synthetic construct
DGSTLYYFDY

SEQ ID NO: 4 - protein, 10 aa, synthetic construct
SASSSVDYMY

SEQ ID NO: 5 - protein, 7 aa, synthetic construct
DTSNLAS

SEQ ID NO: 6 - protein, 10 aa, synthetic construct
QQWSSSPPIT

SEQ ID NO: 7 - protein, 119 aa, synthetic construct
EVMLVESGGALVKPGGSLKLSCAASGFSLSNYVMSWVRQTPEKRLEWVATISSGGRYIYYTDSVKGRFTISRDNARNTLYLQMSSLRSEDTAMYYCARDGSTLYYFDYWGQGTTLTVSS

SEQ ID NO: 8 - protein, 107 aa, synthetic construct
QTVLTQSPAIMSASPGEKVTMTCSASSSVDYMYWYQQKPGSSPRLLIYDTSNLASGVPVRFSGSGSGTSYSLTISRMEAEDAATYYCQQWSSSPPITFGTGTKVELK

SEQ ID NO: 9 - DNA, 357 bp, synthetic construct
  source 1..357: mol_type = other DNA
gaagtgatgctggtggagtctgggggagccttagtgaagcctggagggtccctgaaactctcctgtgcagcttctggattcagtttgagtaactatgtcatgtcttgggttcgccagactccagagaagaggctggagtgggtcgcaaccattagtagtggtggtaggtatatctactatacagacagtgtgaagggtcgattcaccatctccagggacaatgccaggaacaccctgtacctgcaaatgagcagtctgaggtctgaggacacggccatgtattattgtgcaagagacggtagtaccttgtactactttgactattggggccaaggcaccactctcacagtctcctca

SEQ ID NO: 10 - DNA, 321 bp, synthetic construct
  source 1..321: mol_type = other DNA
caaactgttctcacccagtctccagcaatcatgtctgcatctccaggggagaaggtcaccatgacctgcagtgccagctcaagtgtagattacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttccccacccatcacgttcggtactgggaccaaggtggagctgaaa

SEQ ID NO: 11 - protein, 21 aa, synthetic construct
MALPVTALLLPLALLLHAARP

SEQ ID NO: 12 - protein, 21 aa, synthetic construct
MRPRLWLLLAAQLTVLHGNSV

SEQ ID NO: 13 - protein, 15 aa, synthetic construct
GGGGSGGGGSGGGGS

SEQ ID NO: 14 - protein, 241 aa, synthetic construct
EVMLVESGGALVKPGGSLKLSCAASGFSLSNYVMSWVRQTPEKRLEWVATISSGGRYIYYTDSVKGRFTISRDNARNTLYLQMSSLRSEDTAMYYCARDGSTLYYFDYWGQGTTLTVSSGGGGSGGGGSGGGGSQTVLTQSPAIMSASPGEKVTMTCSASSSVDYMYWYQQKPGSSPRLLIYDTSNLASGVPVRFSGSGSGTSYSLTISRMEAEDAATYYCQQWSSSPPITFGTGTKVELK

SEQ ID NO: 15 - protein, 241 aa, synthetic construct
QTVLTQSPAIMSASPGEKVTMTCSASSSVDYMYWYQQKPGSSPRLLIYDTSNLASGVPVRFSGSGSGTSYSLTISRMEAEDAATYYCQQWSSSPPITFGTGTKVELKGGGGSGGGGSGGGGSEVMLVESGGALVKPGGSLKLSCAASGFSLSNYVMSWVRQTPEKRLEWVATISSGGRYIYYTDSVKGRFTISRDNARNTLYLQMSSLRSEDTAMYYCARDGSTLYYFDYWGQGTTLTVSS

SEQ ID NO: 16 - protein, 22 aa, synthetic construct
IWAPLAGTCGVLLLSLVITLYC

SEQ ID NO: 17 - protein, 47 aa, synthetic construct
TTTPAPRPPTPAPTIASQPLSLRPEACRPAAGGAVHTRGLDFACDIY

SEQ ID NO: 18 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 42 aa, synthetic construct
KRGRKKLLYIFKQPFMRPVQTTQEEDGCSCRFPEEEEGGCEL

SEQ ID NO: 20 - protein, 467 aa, synthetic construct
EVMLVESGGALVKPGGSLKLSCAASGFSLSNYVMSWVRQTPEKRLEWVATISSGGRYIYYTDSVKGRFTISRDNARNTLYLQMSSLRSEDTAMYYCARDGSTLYYFDYWGQGTTLTVSSGGGGSGGGGSGGGGSQTVLTQSPAIMSASPGEKVTMTCSASSSVDYMYWYQQKPGSSPRLLIYDTSNLASGVPVRFSGSGSGTSYSLTISRMEAEDAATYYCQQWSSSPPITFGTGTKVELKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 467 aa, synthetic construct
QTVLTQSPAIMSASPGEKVTMTCSASSSVDYMYWYQQKPGSSPRLLIYDTSNLASGVPVRFSGSGSGTSYSLTISRMEAEDAATYYCQQWSSSPPITFGTGTKVELKGGGGSGGGGSGGGGSEVMLVESGGALVKPGGSLKLSCAASGFSLSNYVMSWVRQTPEKRLEWVATISSGGRYIYYTDSVKGRFTISRDNARNTLYLQMSSLRSEDTAMYYCARDGSTLYYFDYWGQGTTLTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 18 bp, synthetic construct
  source 1..18: mol_type = other DNA
ctgggacctcttactctc

SEQ ID NO: 23 - DNA, 18 bp, synthetic construct
  source 1..18: mol_type = other DNA
ccagtaccgaacgtgatg

SEQ ID NO: 24 - DNA, 24 bp, synthetic construct
  source 1..24: mol_type = other DNA
tgtcactcttctcttaggtatgga

SEQ ID NO: 25 - DNA, 20 bp, synthetic construct
  source 1..20: mol_type = other DNA
ggtctttacggatgtcaacg

(19) United States
(12) Patent Application Publication
(10) Pub. No.: US 2025/0288674 A1
(43) Pub. Date: Sep. 18, 2025

(54) CHIMERIC ANTIGEN RECEPTOR AGAINST PROGRAMMED DEATH LIGAND 1 (PD-L1) AND APPLICATION THEREOF

(71) Applicant: TOPMUNNITY THERAPEUTICS TAIWAN LIMITED, Taipei City (TW)

(72) Inventors: Jyun WANG, Taipei City (TW); Chao-Yua HUANG, Taipei City (TW)

(73) Assignee: TOPMUNNITY THERAPEUTICS TAIWAN LIMITED, Taipei City (TW)

(21) Appl. No.: 18/604,880

(22) Filed: Mar. 14, 2024

(51) Int. Cl. A61K 39/00; A61P 35/00; C07K 14/705; C07K 14/725; C07K 16/28; C12N 15/86

(52) U.S. Cl. CPC A61K 40/421; A61K 40/11; A61K 40/31; A61P 35/00; C07K 14/7051; C07K 14/70521; C07K 14/70578; C07K 16/2827; C12N 15/86; C07K 2319/02; C07K 2319/03; C07K 2319/30; C12N 2740/15043

(57) ABSTRACT

Disclosed is a chimeric antigen receptor (CAR) against programmed death ligand 1 (PD-L1) and application thereof. In particular, the CAR comprises an antigen-binding domain which binds to PD-L1; and the anti-PD-L1 CAR-T cells thus produced are useful in CAR cell therapy.

### TECHNOLOGY FIELD

The present invention relates to a chimeric antigen receptor (CAR) against programmed death ligand 1 (PD-L1) and application thereof. In particular, the CAR comprises an antigen-binding domain which binds to PD-L1; and the anti-PD-L1 CAR cells thus produced are useful in CAR cell therapy.

### REFERENCE TO ELECTRONIC SEQUENCE LISTING

The application contains a Sequence Listing which has been submitted electronically in .XML format and is hereby incorporated by reference in its entirety. Said .XML copy; created on Mar. 13, 2024, is named “TMT0001WO” and is 24,828 bytes in size. The sequence listing contained in this .XML file is part of the specification and is hereby incorporated by reference herein in its entirety.

### BACKGROUND OF THE INVENTION

Programmed Death-Ligand 1 (PD-L1) plays a pivotal role in modulating immune responses, both in normal physiological conditions and in cancer. In normal cells, PD-L1 contributes to immune homeostasis by preventing T cell overactivation and protecting against autoimmunity. However, in the context of cancer, PD-L1's overexpression on tumor cells activates the PD-1 pathway on T cells, leading to an impaired immune response and facilitating tumor immune evasion. This dual functionality of PD-L1 underpins its significance as a target in cancer immunotherapy^{1,2}.

The interaction between PD-L1 and PD-1 is intricately influenced by the glycosylation status of PD-L1. Specifically, the deglycosylation at the N35 site on PD-L1 significantly alters its binding affinity with PD-1, impacting the immunosuppressive activity of PD-L1 in cancer^{3,4}. Beyond the N35 site, PD-L1 possesses additional glycosylation sites at N192, N200, and N219, each playing a role in PD-L1's stability and function⁵. These glycosylation sites not only enhance PD-L1's structural integrity but are also crucial for its prolonged interaction with immune cells, contributing to its immune checkpoint functionality.

Studies have revealed that normal tissues and naive immune cells express very low levels of PD-L1 and glycosylated PD-L1 (gPD-L1), highlighting the potential for developing immunotherapies that minimize collateral damage to normal tissues³. Targeting gPD-L1 with specificity could significantly increase the safety profile of these therapies, sparing normal cells that express these molecules at low levels.

Interestingly, FDA-approved anti-PD-L1 antibodies such as Atezolizumab (Tecentriq), Durvalumab (Imfinzi), and Avelumab (Bavencio) demonstrate varying affinities for glycosylated versus deglycosylated forms of PD-L1. This disparity in binding affinity, particularly a reduced affinity for the deglycosylated form of PD-L1, is a critical consideration in the development and clinical application of these antibodies, influencing their effectiveness and specificity in targeting cancer cells⁴.

Chimeric Antigen Receptor T (CAR-T) therapy represents a groundbreaking advancement in cancer treatment, involving the reengineering of a patient's T cells to target and eradicate cancer cells. In a notable instance, a clinical trial involving CAR-T therapy based on Atezolizumab, an anti-PD-L1 antibody, was conducted but terminated early after enrolling only one patient at least due to safety concerns. This innovative approach still faces significant challenges.

### SUMMARY OF THE INVENTION

The present invention provides a chimeric antigen receptor (CAR) against PD-L1, cells expressing the CAR and CAR cell therapy using the same. In particular, the CAR cells as produced exhibit a heightened specific cytotoxicity against PD-L1 expressing cells, and provide superior effects in cancer immunotherapy including a remarkable reduction in tumor growth and persistency of the anti-tumor efficacy.

Particularly, in one aspect, the present invention provides a CAR comprising a PD-L1 binding domain which comprises (a) a heavy chain variable region (V_{H}) which comprises a heavy chain complementary determining region (HC CDR1) of SEQ ID NO: 1, a heavy chain complementary determining region 2 (HC CDR2) of SEQ ID NO: 2, and a heavy chain complementary determining region 3 (HC CDR3) of SEQ ID NO: 3; and (b) a light chain variable region (V_{L}) which comprises a light chain complementary determining region 1 (LC CDR1) of SEQ ID NO: 4, a light chain complementary determining region (LC CDR2) of SEQ ID NO: 5, and a light chain complementary determining region 3 (LC CDR3) of SEQ ID NO: 6.

In some embodiments, the CAR further comprises a hinge domain, a transmembrane domain and an intracellular signaling domain.

In some embodiments, the CAR comprises the PD-L1 binding domain comprising, from N-terminus to C-terminus, the V_{H} and the V_{L} or the V_{L} and the V_{H}.

In some embodiments, the V_{H} and the V_{L} are linked by a linker.

In some embodiments, the intracellular signaling domain comprises at least one be selected from CD137 (4-1BB) signal domain, CD28 signal domain, CD27 signal domain, ICOS signal domain, CD3ζ signal domain and any combination thereof.

In some embodiments, the CAR comprises the amino acid sequence of SEQ ID NO: 14 or 15. In some embodiments, the CAR comprises the amino acid sequence of SEQ ID NO: 20 or 21.

The present invention also provides a nucleic acid molecule comprising a nucleotide sequence encoding a CAR as described herein. In some examples, the nucleic acid molecule is a vector.

The present invention also provides a cell comprising a nucleic acid molecule comprising a nucleotide sequence encoding a CAR as described herein. The cells are engineered to express the CAR on their surface which are useful in CAR cell therapy. A pharmaceutical composition comprising a cell for expressing a CAR as described herein is also provided.

In another aspect, the present invention also provides a method for treating a subject having a disease, disorder or condition associated with an elevated expression of a tumor antigen by administering to the subject an effective amount of a cell genetically modified to express a CAR as described herein. The present invention also provides use of a cell genetically modified to express a CAR as described herein for manufacturing a medicament for treating a subject having a disease, disorder or condition associated with an elevated expression of a tumor antigen.

In some embodiments, the tumor antigen is PD-L1.

In some embodiments, the subject is suffered from cancer.

In some embodiments, the cancer is selected from the group consisting of breast cancer, colon cancer, head and neck cancer, thyroid cancer, lung cancer, ovarian cancer, pancreatic cancer, gastric cancer, kidney cancer, glioblastoma and leukemia.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows the structure of CAR in the viral vector backbone plasmid. pTT0001 comprises a human CD8 signal peptide, myc, V_{H} of mTT-01, GS linker, V_{L} of mTT-01, human CD8a transmembrane domain, 4-1BB, and human CD3ζ; pTT0002 comprises a human CD8 signal peptide, myc, V_{L} of mTT-01, GS linker, V_{H} of mTT-01, human CD8a transmembrane domain, 4-1BB, and human CD3ζ. Herein, myc serves as a subsequent experimental marker for CAR proteins. Before the human CD8a transmembrane domain, we add Alanine-Alanine-Alanine as a NotI restriction enzyme cutting site.

FIG. 2 shows a comparison of immunohistochemical staining between the monoclonal antibody mTT-01 and chimeric Atezolizumab in human placental tissue. The left image shows the staining result of mTT-01; the right image shows the staining result of chimeric Atezolizumab. The placenta, being fetal tissue, thus expresses higher levels of PD-L1 to prevent attack by maternal T cells. The arrows indicate that both antibodies clearly stain the cell membranes.

FIG. 3 shows a comparative immunohistochemical staining of the monoclonal antibody mTT-01 and chimeric Atezolizumab in human tonsillar tissue. The left image presents the staining result of mTT-01; the right image, that of chimeric Atezolizumab. The tonsil, a human organ tissue, exhibits higher PD-L1 expression. Both antibodies show a similar staining distribution, but the staining by mTT-01 is noticeably weaker than chimeric Atezolizumab.

FIG. 4 shows the comparative immunohistochemical staining of the monoclonal antibody mTT-01 and chimeric Atezolizumab in human breast cancer tumors. The left image shows the staining result of mTT-01; the right image, that of chimeric Atezolizumab staining result. Both antibodies display similar staining distribution, with chimeric Atezolizumab showing deeper staining in the cytoplasm, and mTT-01 showing deeper staining on the tumor cell membranes. Additionally, chimeric Atezolizumab shows staining in the stroma and nucleus of the breast tissue.

FIG. 5 shows a comparison of immunohistochemical staining between the monoclonal antibody mTT-01 and chimeric Atezolizumab in human tumors and adjacent normal tissues. Given the importance of CAR technology in enabling immune cells to recognize biomarkers on the cell surface, out of 43 tumor samples with cell membrane staining as positive, mTT-01 recognized 34 samples, yielding a positive recognition rate of 79%; chimeric Atezolizumab recognized 20 samples, with a positive recognition rate of 46.5%. Among 44 samples of tumor-adjacent normal tissue, mTT-01 showed cell membrane staining in only 3 samples, adjacent normal tissue recognition rate of 6.8%; chimeric Atezolizumab showed staining in 10 samples, adjacent normal tissue recognition rate of 22.7%. This indicates that mTT-01 has better specificity than chimeric Atezolizumab in cell membrane staining, potentially leading to lower toxicity to normal tissues.

FIG. 6 shows Jurkat NFAT-Luc cells, a T lymphocyte-derived immortal cell line, which integrate an NFAT (Nuclear Factor of Activated T-Cells)-inducible Lucia reporter, leading to the expression of luciferase upon activation of the NFAT pathway. Following transduction of TT0001 and TT0002 CAR into Jurkat NFAT-Luc cells using lentiviral vectors, myc expression can be detected on the Jurkat cell surface. The left image represents normal Jurkat NFAT-Luc cells, used to determine the normal range of myc expression, with the distribution of cells exceeding the threshold marked at 0.22%; the middle image shows the distribution of myc in TT0001 CAR-Jurkat cells using the same threshold, revealing 81.9% of cells expressing myc; the right image shows the distribution of myc in TT0002 CAR-Jurkat cells using the same threshold, revealing 78.6% of cells expressing myc.

FIG. 7 shows co-culture of CAR-Jurkat cells with four types of cells: MDA-MB-231, a breast cancer cell line with high PD-L1 expression; BT549, a breast cancer cell line with medium PD-L1 expression; A549, a lung cancer cell line with low PD-L1 expression; HEK293T, a human embryonic kidney cell, not expressing PD-L1. When Jurkat cells are induced to activate NFAT, they produce luciferase, detectable by bioluminescence. Within two days, normal Jurkat group shows extremely low bioluminescence, while TT0001 CAR-Jurkat and TT0002 CAR-Jurkat display bioluminescence intensity positively correlated with the PD-L1 expression on the surface of co-cultured cells. This confirms that CAR-Jurkat, when co-cultured with target cancer cells, can correctly activate the NFAT pathway, demonstrating the proper function of the CAR's signal peptide.

FIG. 8 shows establishment of CAR constructs in CD3+ T cells, TT0001 CAR-T. After infecting primary CD3+ T cells with a lentiviral vector, the surface expression of myc is detected using FACS. The upper middle image shows normal CD3+ T cells, used to determine the normal range of myc expression, outlining a cell distribution exceeding the threshold at 0.74%. The embodiment used three different lentivirus infection concentrations, dividing the populations with the same threshold to observe that over 78% of T cells express myc, with an MFI of around 3000, significantly higher than the 33.7 MFI of normal CD3+ T cells, indicating successful expression of TT0001 in CAR-T cells.

FIG. 9 shows establishment of CAR constructs in CD3+ T cells, TT0002 CAR-T. After infecting primary CD3+ T cells with a lentiviral vector, the surface expression of myc is detected using FACS. The upper middle image shows normal CD3+ T cells, used to determine the normal range of myc expression, outlining a cell distribution exceeding the threshold at 0.74%. The embodiment used three different lentivirus infection concentrations, dividing the populations with the same threshold to observe that over 79% of T cells express myc, with an MFI of around 3000, significantly higher than the 33.7 MFI of normal CD3+ T cells, indicating successful expression of TT0002 in CAR-T cells.

FIG. 10 shows cytotoxicity analysis in breast cancer cell lines with CAR-T Treatment. The top panel shows the cytotoxicity of TT0001 and TT0002 CAR-T cells against MDA-MB-231 breast cancer cells, measured using the CCK-8 assay after 24 hours of co-culture. The results demonstrate that both CAR-T variants exhibit similar cytotoxic capabilities without significant differences, with their efficacy increasing alongside the E:T ratio. At an E:T ratio of 1:3, approximately 20% cytotoxicity was observed; at 1:1, cytotoxicity increased to about 60%; and at ratios above 3:1, over 80% cytotoxicity was achieved. In contrast, control CD3+ T cells only displayed noticeable cytotoxic effects at an E:T ratio of 10:1. The bottom panel shows cytotoxicity of TT0002 CAR-T in various breast cancer cell lines. TT0002 CAR-T cells exhibited varying degrees of cytotoxicity across multiple breast cancer cell lines. Notably, moderate cytotoxicity was observed in the MDA-MB-361 cell line. The remaining cell lines demonstrated a high level of cytotoxic response to TT0002 CAR-T.

FIG. 11 shows MDA-MB-231 cell lines exhibiting varying levels of PD-L1 expression. The MDA-MB-231 PD-L1 cell line overexpresses PD-L1; the MDA-MB-231 cell line expresses PD-L1 at a high level; the MDA-MB-231 PD-L1^{KO} cell line does not express PD-L1.

FIG. 12 shows divergent cytotoxicity of TT0002 CAR-T against MDA-MB-231 derived cell lines with varied PD-L1 expression. The top panel shows TT0002 CAR-T cells displaying different levels of cytotoxic effects against the MDA-MB-231 cell line series, depending on the PD-L1 expression levels. The degree of cytotoxicity was found to be positively correlated with the amount of PD-L1 expression, as well as with the number of effective cells. The bottom panel shows the results that CD3+ T cells displaying no significant cytotoxicity.

FIG. 13 shows a Multi-Plex assay to analyze the cytokine levels released into the medium after a 48-hour co-culture of TT0002 CAR-T cells with MDA-MB-231 cell lines exhibiting varying levels of PD-L1 expression. The effector cells are the TT0002 CAR-T cells, and the target cells are the MDA-MB-231 cell lines. The upper-left graph shows the results for human IL-2, with no detection of IL-2 in the medium for MDA-MB-231 PD-L1^{KO} co-cultures, regardless of the E:T ratio. For the other two cell lines, the highest concentration of IL-2 in the medium is detected at an E:T ratio of 3:1, decreasing at lower ratios, with the MDA-MB-231 PD-L1 cell line exhibiting a higher expression of IL-2. Although IL-2 is detected at the highest E:T ratio of 10:1, its concentration is lower than in the 3:1 group because of consumption by TT0002 CAR-T, with the MDA-MB-231 PD-L1 cell line having a slightly lower IL-2 concentration than the MDA-MB-231 cell line. This indicates that IL-2 is produced by the interaction of TT0002 CAR-T cells with cells expressing PD-L1 on their surface, with a higher expression correlating with an increased number of T cells and generally being positively related to the PD-L1 expression level. The middle-upper graph shows the results for human TNF-α, with no detection of TNF-α in the medium for MDA-MB-231 PD-L1^{KO} co-cultures, regardless of the E:T ratio. For the other two cell lines, the highest concentration of TNF-α in the medium is detected at an E:T ratio of 3:1, decreasing at lower ratios, with the MDA-MB-231 PD-L1 cell line exhibiting a higher expression of TNF-α. Although TNF-α is detected at the highest E:T ratio of 10:1, its concentration is lower than in the 3:1 group. This indicates that TNF-α is produced by the interaction of TT0002 CAR-T cells with cells expressing PD-L1 on their surface, with a higher expression correlating with an increased number of T cells and generally being positively related to the PD-L1 expression level. The upper-right graph shows the results for human IFN-γ, with the highest concentration of IFN-γ in the medium detected at an E:T ratio of 3:1, decreasing at lower ratios. Within each E:T group, the difference between MDA-MB-231 PD-L1 and MDA-MB-231 is not significant, with MDA-MB-231 PD-L1^{KO} detecting a small amount of IFN-γ; the highest E:T ratio of 10:1 detects an IFN-γ concentration close to but lower than the 3:1 group. This indicates that IFN-γ is produced by the interaction of TT0002 CAR-T cells with MDA-MB-231 co-culture cells, with a higher expression correlating with an increased number of T cells, and a high expression of PD-L1 on the cell surface significantly stimulating the production of IFN-γ. The lower-left graph shows the results for human IL-10, with the highest concentration of IL-10 in the medium detected at an ET ratio of 10:1, decreasing at lower ratios. Within each E:T group, the MDA-MB-231 PD-L1 cell line has a higher IL-10 concentration than the other two cell lines at high ratios, with MDA-MB-231 PD-L1^{KO} detecting a lesser amount of IL-10. This indicates that IL-10 is produced by the interaction of TT0002 CAR-T cells with MDA-MB-231 co-culture cells, with a higher expression correlating with an increased number of T cells, and a high expression of PD-L1 on the cell surface significantly stimulating the production of IL-10 at high E:T ratios. The middle-lower graph shows the results for human IL-6. From the group not co-cultured with TT0002 CAR-T cells, it can be seen that both MDA-MB-231 PD-L1 and MDA-MB-231 cells have a high expression of IL-6, indicating that the 231 cell lines themselves produce IL-6; apart from the MDA-MB-231 PD-L1^{KO} group, which only expresses IL-6 after co-culture, the other two groups see a decrease following an increase in T cells.

FIG. 14 shows the in vivo efficacy of TT0001 CAR-T and TT0002 CAR-T. NSG mice are subcutaneously injected with 1×10⁷ MDA-MB-231 cells as the tumor model, with the day of injection designated as day 0. On day 15, when the tumor exceeds 100 mm³, 1×10⁷ TT0001 CAR-T cells are intravenously injected, with a two-dose regimen receiving a second intravenous injection of 1×10⁷ TT0001 CAR-T cells one week later; the control group is injected with PBS on day 15. The TT0002 CAR-T group waits until day 21 when the tumor exceeds 200 mm³ to intravenously inject 1×10⁷ TT0002 CAR-T cells, followed by a second dose of CAR-T three days later; the control group is injected with PBS on day 15. The mouse's weight and tumor size are measured every three days. The experiment concludes with the sacrifice of the mice.

FIG. 15 shows the therapeutic efficacy of TT0001 CAR-T and TT0002 CAR-T in the xenograft mouse model. The data shows that with an injection of TT0001 CAR-T even the tumor is smaller, there is no observable tumor suppression effect; however, with TT0002 CAR-T, tumor size is suppressed to 300 mm³ until day 52.

FIG. 16 shows the therapeutic efficacy of TT0002 CAR-T in the xenograft mouse model for an extended period of time. The data shows that TT0002 CAR-T extends the tumor growth period, reducing the MDA-MB-231 injection to 5×10⁶ and similarly waiting until the tumor exceeds 100 mm³ before injecting 1×10⁷ TT0002 CAR-T, followed by a second dose of 1×10⁷ TT0002 CAR-T three days later. The control group is injected with PBS at the same time points. Body weight and tumor size are measured every two to three days until the mice die or are sacrificed. The lower graph shows that the TT0002 CAR-T injected group significantly suppresses tumor growth, with tumors undetectable around day 30; on day 24, one mouse injected with CAR-T is sacrificed, and one dies, with the remaining mice living until day 44 when the control group is sacrificed, leaving two mice for subsequent rechallenge experiments.

FIG. 17 shows the dose-dependent tumor suppression effect of TT0002 CAR-T. The upper-left graph shows the experimental design, with 3×10⁶ MDA-MB-231-Luc cells subcutaneously injected on day 0, followed by an intravenous injection of one dose of T cells (1×10⁷ CD3+ T, 1×10⁷ TT0002 CAR-T, or 1×10⁶ TT0002 CAR-T) on day 14. The lower-left graph shows different tumor suppression effects for different doses of CAR-T; CD3+ T cells do not suppress the tumor; 1×10⁶ TT0002 CAR-T maintains tumor size until around day 28 no longer suppressing it; 1×10⁷ TT0002 CAR-T suppresses the tumor until it nearly disappears by day 42. The right graph shows the growth of MDA-MB-231-Luc tumors in mice, with both the 1×10⁷ CD3+ T and 1×10⁶ TT0002 CAR-T groups showing tumor metastasis by day 50, with only the 1×10⁷ TT0002 CAR-T group showing tumor shrinkage or disappearance.

FIG. 18 shows the sustained efficacy of TT0002 CAR-T in the rechallenged xenograft mouse model. The experiment continues the aforementioned experiment which describe in FIG. 16, rechallenging the two surviving mice with 1×10⁷ MDA-MB-231 tumor injection and sacrificing them on day 90. Body weight and tumor size at the injection site are measured every two to three days. The graph below shows that, compared to the control group injected with tumors at the same time, mice injected with TT0002 CAR-T do not develop tumors, proving that CAR-T remains in the mice for at least 44 days and still has tumor suppression efficacy.

FIG. 19 shows the distribution of CAR-T cells in various tissues. The test analyzes blood, spleen, and bone marrow tissues extracted from rechallenge mice using FACS, with live & dead signals as the vertical axis and human CD3 as the horizontal axis. Using the threshold outlined in the upper-left graph of cultured TT0002 CAR-T, cells falling into the Q3 region are identified as TT0002 CAR-T, observing whether CAR-T is maintained in the tissues. As indicated by the arrows, only a very small amount of CAR-T (<1%) is detected in the blood and spleen, with a larger amount of CAR-T (3.18%) found only in the bone marrow.

FIG. 20 shows the sustained efficacy of TT0002 CAR-T in the rechallenged xenograft mouse model. The experiment extends the tumor rechallenge experiment to day 86 with the administration of MDA-MB-231 cells. It is observed that although tumor growth continues to be suppressed, complete prevention of tumor growth is not achieved. This indicates the presence of CAR-T cells within the mouse body on day 86, though in significantly reduced numbers compared to day 44.

FIG. 21 shows penetration and activation of TT0002 CAR-T in xenograft tumors. The experiment employs immunohistochemistry to confirm the presence of CAR-T cells in mouse xenograft tumor tissues. The top row images are from the PBS-injected control group mouse tumor sections, and the bottom row images are from the TT-0002 CAR-T-injected mouse tumor sections. In the control group, widespread PD-L1 expression is evident in the tissues (top right image, red area), but cells expressing human CD3 are absent (top middle image, red area). In contrast, the TT0002 CAR-T group shows a ring of human CD3-expressing CAR-T cells around the central blood vessels in the tissue (bottom middle image, central red area), with the outer ring being PD-L1 high-expressing tumor tissue (bottom right image, red area). The top right section of the slice shows the simultaneous presence of PD-L1 and human CD3. This confirms that CAR-T cells are indeed attracted to the tumor.

FIG. 22 shows the presence and activity of TT0002 CAR-T in xenograft tumors. The experiment utilizes immunofluorescence staining to verify the presence of CAR-T cells in mouse xenograft tumor tissues. The top row images are from the PBS-injected control group mouse tumor sections, and the bottom row images are from the TT-0002 CAR-T-injected mouse tumor sections. The first column on the left shows nuclear staining of the two groups of tissue sections; the second column on the left shows human CD3 staining, representing CAR-T; the middle column shows human PD-L1 staining, representing tumor cells; the second column on the right shows Granzyme B staining, released by T cells, indicating CAR-T action on target tumor cells; the far-right column shows false-color overlays of all stains. In the control group, extensive PD-L1 expression is visible (top middle image), but no human CD3 expression is observed (top second from the left image), and almost no signal of T cell killing of tumor cells is seen (top second from the right image). In contrast, the TT0002 CAR-T group shows hCD3 signals (bottom second from the left image) colocalized with hPD-L1 signals (bottom middle image), along with a substantial presence of Granzyme B signals (bottom second from the right image), demonstrating that CAR-T cells can infiltrate into tumor tissue and kill tumor cells.

FIG. 23 shows the biodistribution and migration of TT0002 CAR-T the xenograft mouse model. It pertains to the biodistribution experiment of TT0002 CAR-T, confirming its primary distribution in the lungs and tumors of mice during CAR-T treatment. The top image shows the tumor growth curves of each mouse before sacrifice, with tumors disappearing after day 35 (21 days post CAR-T injection). The bottom image shows the CAR copy content in DNA samples taken at various time points, approximating the amount of CAR-T cells in the sample. It is observed that within 72 hours post CAR-T injection, CAR-T cells are mainly distributed in the lungs, and after seven days, they are primarily located in the tumor.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

### I. General Definitions

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, 20 or 10 amino acid residues).

As used herein, the term “approximately” or “about” refers to a degree of acceptable deviation that will be understood by persons of ordinary skill in the art, which may vary to some extent depending on the context in which it is used. Specifically, “approximately” or “about” may mean a numeric value having a range of ±10% or ±5% or ±3% around the cited value.

As used herein, the term “substantially identical” refers to two sequences having 80% or more, preferably 85% or more, more preferably 90% or more, even more preferably 95% or more homology.

As used herein, the term “antibody” (interchangeably used in plural form, antibodies) means an immunoglobulin molecule having the ability to specifically bind to a particular target antigenic molecule. As used herein, the term “antibody” includes not only intact (i.e. full-length) antibody molecules but also antigen-binding fragments thereof retaining antigen binding ability e.g. Fab, Fab′, F(ab′)2 and Fv. Such fragments are also well known in the art and are regularly employed both in vitro and in vivo. The term “antibody” also includes chimeric antibodies, humanized antibodies, human antibodies, diabodies, linear antibodies, single chain antibodies, multispecific antibodies (e.g., bispecific antibodies) and any other modified configuration of the immunoglobulin molecule that comprises an antigen recognition site of the required specificity, including amino acid sequence variants of antibodies, glycosylation variants of antibodies, and covalently modified antibodies.

An intact or complete antibody comprises two heavy chains and two light chains. Each heavy chain contains a variable region (V_{H}) and a first, second and third constant regions (C_{H}1, C_{H}2 and C_{H}3); and each light chain contains a variable region (V_{L}) and a constant region (C_{L}). The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light chains and those of heavy chains are responsible for antigen binding. The variables regions in both chains are responsible for antigen binding generally, each of which contain three highly variable regions, called the complementarity determining regions (CDRs); namely, heavy (H) chain CDRs including HC CDR1, HC CDR2, HC CDR3 and light (L) chain CDRs including LC CDR1, LC CDR2, and LC CDR3. The three CDRs are franked by framework regions (FR1, FR2, FR3, and FR4), which are more highly conserved than the CDRs and form a scaffold to support the hypervariable regions. The constant regions of the heavy and light chains are not responsible for antigen binding, but involved in various effector functions. Depending on the antibody amino acid sequence of the constant domain of its heavy chains, immunoglobulins can be assigned to different classes. There are five major classes of immunoglobulins: IgA, IgD, IgE, IgG, and IgM. The heavy-chain constant domains that correspond to the different classes of immunoglobulins are called alpha, delta, epsilon, gamma, and mu, respectively.

As used herein, the term “antigen-binding fragment” or “antigen-binding domain” refers to a portion or region of an intact antibody molecule that is responsible for antigen binding. An antigen-binding fragment is capable of binding to the same antigen to which the parent antibody binds. Examples of antigen-binding fragments include, but are not limited to: (i) a Fab fragment, which can be a monovalent fragment composed of a V_{H}-C_{H}1 chain and a V_{L}-C_{L} chain; (ii) a F(ab′)2 fragment which can be a bivalent fragment composed of two Fab fragments linked by a disulfide bridge at the hinge region; (iii) a Fv fragment, composed of the V_{H} and V_{L} domains of an antibody molecule associated together by noncovalent interaction; (iv) a single chain Fv (scFv), which can be a single polypeptide chain composed of a V_{H} domain and a V_{L} domain via a peptide linker; and (v) a (scFv)₂, which can contain two V_{H} domains linked by a peptide linker and two V_{L} domains, which are associated with the two Vu domains via disulfide bridges.

As used herein, the term “chimeric antibody” refers to an antibody containing polypeptides from different sources, e.g., different species. In some embodiments, in chimeric antibodies, the variable region of both light and heavy chains may mimic the variable region of antibodies derived from one species of mammal (e.g., a non-human mammal such as mouse, rabbit and rat), while the constant region may be homologous to the sequences in antibodies derived from another mammal such as a human.

As used herein, the term “humanized antibody” refers to an antibody comprising a framework region originated from a human antibody and one or more CDRs from a non-human (usually a mouse or rat) immunoglobulin.

As used herein, the term “human antibody” refers to an antibody in which essentially the entire sequences of the light chain and heavy chain sequences, including the complementary determining regions (CDRs), are from human genes. In some circumstances, the human antibodies may include one or more amino acid residues not encoded by human germline immunoglobulin sequences e.g. by mutations in one or more of the CDRs, or in one or more of the FRs, such as to, for example, decrease possible immunogenicity, increase affinity, and eliminate cysteines that might cause undesirable folding, etc.

As used herein, the term “specific binds” or “specifically binding” refers to a non-random binding reaction between two molecules, such as the binding of the antibody to an epitope of its target antigen. An antibody that “specifically binds” to a target antigen or an epitope is a term well understood in the art, and methods to determine such specific binding are also well known in the art. An antibody “specifically binds” to a target antigen if it binds with greater affinity/avidity, more readily, and/or greater duration than it binds to other substances. In other words, it is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, the affinity of the binding can be defined in terms of a dissociation constant (K_{D}). Typically, specifically binding when used with respect to an antibody can refer to an antibody that specifically binds to (recognize) its target with an KD value less than about 10⁻⁷ M, such as about 10⁻⁸ M or less, such as about 10⁻⁹ M or less, about 10⁻¹⁰ M or less, about 10⁻¹¹ M or less, about 10⁻¹² M or less, or even less, and binds to the specific target with an affinity corresponding to a K_{D} that is at least ten-fold lower than its affinity for binding to a non-specific antigen (such as BSA or casein), such as at least 100 fold lower, e.g. at least 1,000 fold lower or at least 10,000 fold lower.

As used herein, the term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

As used herein, the term “complementary” refers to the topological compatibility or matching together of interacting surfaces of two polynucleotides. A first polynucleotide is complementary to a second polynucleotide when the nucleotide sequence of the first polynucleotide is identical to the nucleotide sequence of the polynucleotide binding partner of the second polynucleotide. Thus, the polynucleotide whose sequence 5′-GATAT-3′ is complementary to a polynucleotide whose sequence is 5′-ATATC-3′.”

As used herein, the term “encoding” refers to the natural property of specific sequences of nucleotides in a polynucleotide (e.g., a gene, a cDNA, or an mRNA) to serve as templates for synthesis of other polymers and macromolecules in biological processes having either a given sequence of RNA transcripts (i.e., rRNA, RNA and mRNA) or a given sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” encompasses all nucleotide sequences that are degenerate versions of each other and that encode the same amino acid sequence.

As used herein, the term “recombinant nucleic acid” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence (expression vector) or maintaining the given nucleotide sequence for replicating it, manipulating it or transferring it between different locations (e.g., between different organisms). Vectors can be introduced into a suitable host cell for the above-described purposes. A “recombinant cell” refers to a host cell that has had introduced into it a recombinant nucleic acid. “A transformed cell” mean a cell into which has been introduced, by means of recombinant DNA techniques, a DNA molecule encoding a protein of interest.

Vectors may be of various types, including plasmids, cosmids, episomes, fosmids, artificial chromosomes, phages, viral vectors, etc. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprise, for example and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, a secretion signal sequence (e.g., α-mating factor signal), a stop codon, and other control sequence (e.g., Shine-Dalgarno sequences and termination sequences). Preferably, vectors may further contain a marker sequence (e.g., an antibiotic resistant marker sequence) for the subsequent screening/selection procedure. For purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes a tag for purpose of purification e.g. a His-tag.

As used herein, the term “treatment” refers to the application or administration of one or more active agents to a subject afflicted with a disorder, a symptom or condition of the disorder, or a progression of the disorder, with the purpose to cure, heal, alleviate, relieve, alter, remedy, ameliorate, improve, or affect the disorder, the symptom or condition of the disorder, the disabilities induced by the disorder, or the progression or predisposition of the disorder.

### II. Antibodies Against PD-L1

The present invention is based, at least in part, on antibodies against PD-L1, for preparing a chimeric antigen receptor (CAR). The anti-PD-L1 antibodies as used herein are found to specifically target PD-L1 at certain glycosylation sites. The anti-PD-L1 antibodies as used herein are useful in developing a new CAR construct aiming to improve the efficacy of CAR-T therapy.

Exemplary anti-PD-L1 antibodies are as described in U.S. Pat. No. 11,660,352 B2, the relevant disclosures of which are incorporated by reference herein for the purposes or subject matter referenced herein. One particular example of the anti-PD-L1 antibodies comprises a heavy chain variable region (V_{H}) having complementary determining regions thereof (HC CDR1, HC CDR2, and HC CDR3) and a light chain variable region (V_{L}) having complementary determining regions thereof (LC CDR1, LC CDR2, and LC CDR3) as shown in Table 1 below.

TABLE 1
Amino acid sequences of anti-PD-L1 antibody,
mTT-01

| Anti-PD-L1 | |
|---|---|
| antibody, | |
| mTT-01 | Amino acid sequence |
| Heavy Chain | NYVMS |
| CDR1 | (SEQ ID NO: 1) |
| Heavy Chain | TISSGGRYIYYTDSVKG |
| CDR2 | (SEQ ID NO: 2) |
| Heavy Chain | DGSTLYYFDY |
| CDR3 | (SEQ ID NO: 3) |
| V_{H} Domain | EVMLVESGGALVKPGGSLKLSCAASGFSLS |
| | NYVMSWVRQTPEKRLEWVATISSGGRYIYY |
| | TDSVKGRFTISRDNARNTLYLQMSSLRSED |
| | TAMYYCARDGSTLYYFDYWGQGTTLTVSS |
| | (SEQ ID NO: 7) |
| Light Chain | SASSSVDYMY |
| CDR1 | (SEQ ID NO: 4) |
| Light Chain | DTSNLAS |
| CDR2 | (SEQ ID NO: 5) |
| Light Chain | QQWSSSPPIT |
| CDR3 | (SEQ ID NO: 6) |
| V_{L} Domain | QTVLTQSPAIMSASPGEKVTMTCSASSSVD |
| | YMYWYQQKPGSSPRLLIYDTSNLASGVPVR |
| | FSGSGSGTSYSLTISRMEAEDAATYYCQQW |
| | SSSPPITFGTGTKVE |
| | LK |
| | (SEQ ID NO: 8) |

TABLE 2
DNA sequences of anti-PD-L1 antibody, mTT-01
Anti-PD-L1 antibody, mTT-01

V_{H} Domain
GAAGTGATGCTGGTGGAGTCTGGGGGAGCCTTAGTGAAGCCTGGAGGGTC
CCTGAAACTCTCCTGTGCAGCTTCTGGATTCAGTTTGAGTAACTATGTCA
TGTCTTGGGTTCGCCAGACTCCAGAGAAGAGGCTGGAGTGGGTCGCAACC
ATTAGTAGTGGTGGTAGGTATATCTACTATACAGACAGTGTGAAGGGTCG
ATTCACCATCTCCAGGGACAATGCCAGGAACACCCTGTACCTGCAAATGA
GCAGTCTGAGGTCTGAGGACACGGCCATGTATTATTGTGCAAGAGACGGT
AGTACCTTGTACTACTTTGACTATTGGGGCCAAGGCACCACTCTCACAGT
CTCCTCA (SEQ ID NO: 9)
V_{L} Domain
CAAACTGTTCTCACCCAGTCTCCAGCAATCATGTCTGCATCTCCAGGGGA
GAAGGTCACCATGACCTGCAGTGCCAGCTCAAGTGTAGATTACATGTACT
GGTACCAGCAGAAGCCAGGATCCTCCCCCAGACTCCTGATTTATGACACA
TCCAACCTGGCTTCTGGAGTCCCTGTTCGCTTCAGTGGCAGTGGGTCTGG
GACCTCTTACTCTCTCACAATCAGCCGAATGGAGGCTGAAGATGCTGCCA
CTTATTACTGCCAGCAGTGGAGTAGTTCCCCACCCATCACGTTCGGTACT
GGGACCAAGGTGGAGCTGAAA (SEQ ID NO: 10)

In some embodiments, the anti-PD-L1 antibody is a functional variant of mTT-01 which is characterized in comprising (a) a V_{H} comprising HC CDR1 of SEQ ID NO: 1, HC CDR2 of SEQ ID NO: 2, and HC CDR3 of SEQ ID NO: 3; and (b) a V_{L} comprising LC CDR1 of SEQ ID NO: 4, LC CDR2 of SEQ ID NO: 5, and HC CDR3 of SEQ ID NO: 6. In some embodiments, the anti-PD-L1 antibody can comprise a V_{H} comprising SEQ ID NO: 7 or an amino acid sequence substantially identical thereto and a V_{L} comprising SEQ ID NO: 8 or an amino acid sequence substantially identical thereto. Specifically, the anti-PD-L1 antibody of the present invention includes a V_{H} comprising an amino acid sequence has at least 80% (e.g. 82%, 84%, 85%, 86%, 88%, 90%, 92%, 94%, 95%, 96%, 98%, or 99%) identity to SEQ ID NO:7, and a V_{L} comprising an amino acid sequence has at least 80% (e.g. 82%, 84%, 85%, 86%, 88%, 90%, 92%, 94%, 95%, 96%, 98%, or 99%) identity to SEQ ID NO:8. The anti-PD-L1 antibody of the present invention also includes any recombinantly (engineered)-derived antibody encoded by the polynucleotide sequence encoding the relevant V_{H} or V_{L} amino acid sequences as described herein.

The term “substantially identical” can mean that the relevant amino acid sequences (e.g., in FRs, CDRs, V_{H}, or V_{L}) of a variant differ insubstantially as compared with a reference antibody such that the variant has substantially similar binding activities (e.g., affinity, specificity, or both) and bioactivities relative to the reference antibody. Such a variant may include minor amino acid changes. It is understandable that a polypeptide may have a limited number of changes or modifications that may be made within a certain portion of the polypeptide irrelevant to its activity or function and still result in a variant with an acceptable level of equivalent or similar biological activity or function. In some examples, the amino acid residue changes are conservative amino acid substitution, which refers to the amino acid residue of a similar chemical structure to another amino acid residue and the polypeptide function, activity or other biological effect on the properties smaller or substantially no effect. Typically, relatively more substitutions can be made in FR regions, in contrast to CDR regions, as long as they do not adversely impact the binding function and bioactivities of the antibody (such as reducing the binding affinity by more than 50% as compared to the original antibody). In some embodiments, the sequence identity can be about 80%, 82%, 84%, 85%, 86%, 88%, 90%, 92%, 94%, 95%, 96%, 98%, or 99%, or higher, between the reference antibody and the variant. Variants can be prepared according to methods for altering polypeptide sequence known to one of ordinary skills in the art such as those found in references which compile such methods, e.g., Molecular Cloning: A Laboratory Manual, J. Sambrook, et al., eds., Second Edition, Cold Spring Harbor Laboratory Press, Cold Spring Harbor, New York, 1989. For example, conservative substitutions of amino acids include substitutions made amongst amino acids within the following groups: (i) A, G; (ii) S, T; (iii) Q, N; (iv) E, D; (v) M, I, L, V; (vi) F, Y, W; and (vii) K, R, H.

The antibodies described herein may be animal antibodies (e.g., mouse-derived antibodies), chimeric antibodies (e.g., mouse-human chimeric antibodies), humanized antibodies, or human antibodies. Antigen-binding fragments of the antibodies described herein include a Fab fragment, a F(ab′)2 fragment, a Fv fragment, a single chain Fv (scFv) and a (scFv)₂. The antibodies or their antigen-binding fragments can be prepared by methods known in the art.

### Iii. Preparation of Antibodies or Antigen-Binding Fragments Thereof

Numerous methods conventional in this art are available for obtaining antibodies or antigen-binding fragments thereof.

In some embodiments, the antibodies provided herein may be made by the conventional hybridoma technology. In general, a target antigen optionally coupled to a carrier protein, e.g. keyhole limpet hemocyanin (KLH), and/or mixed with an adjuvant, e.g. complete Freund's adjuvant, may be used to immunize a host animal for generating antibodies binding to that antigen. Lymphocytes secreting monoclonal antibodies are harvested and fused with myeloma cells to produce hybridoma. Hybridoma clones formed in this manner are then screened to identify and select those that secrete the desired monoclonal antibodies.

In some embodiments, the antibodies provided herein may be prepared via recombinant technology. In related aspects, isolated nucleic acids that encode the disclosed amino acid sequences, together with vectors comprising such nucleic acids and host cells transformed or transfected with the nucleic acids, are also provided.

For examples, nucleic acids comprising nucleotide sequences encoding the heavy and light chain variable regions of such an antibody can be cloned into expression vectors (e.g., a bacterial vector such as an E. coli vector, a yeast vector, a viral vector, or a mammalian vector) via routine technology, and any of the vectors can be introduced into suitable cells (e.g., bacterial cells, yeast cells, plant cells, or mammalian cells) for expression of the antibodies. Examples of nucleotide sequences encoding the heavy and light chain variable regions of the antibodies as described herein are as shown in Table 2. Examples of mammalian host cell lines are human embryonic kidney line (293 cells), baby hamster kidney cells (BHK cells), Chinese hamster ovary cells (CHO cells), African green monkey kidney cells (VERO cells), and human liver cells (Hep G2 cells). The recombinant vectors for expression the antibodies described herein typically contain a nucleic acid encoding the antibody amino acid sequences operably linked to a promoter, either constitutive or inducible. Typical vectors contain transcription and translation terminators, initiation sequences, and promoters useful for regulation of the expression of the nucleic acid encoding the antibody. The vectors optionally contain selection markers for both prokaryotic and eukaryotic systems. In some examples, both the heavy and light chain coding sequences are included in the same expression vector. In other examples, each of the heavy and light chains of the antibody is cloned into an individual vector and produced separately, which can be then incubated under suitable conditions for antibody assembly.

The recombinant vectors for expression the antibodies described herein typically contain a nucleic acid encoding the antibody amino acid sequences operably linked to a promoter, either constitutive or inducible. The recombinant antibodies can be produced in prokaryotic or eukaryotic expression systems, such as bacteria, yeast, insect and mammalian cells. Typical vectors contain transcription and translation terminators, initiation sequences, and promoters useful for regulation of the expression of the nucleic acid encoding the antibody. The vectors optionally contain selection markers for both prokaryotic and eukaryotic systems. The antibody protein as produced can be further isolated or purified to obtain preparations that substantially homogeneous for further assays and applications. Suitable purification procedures, for example, may include fractionation on immunoaffinity or ion-exchange columns, ethanol precipitation, sodium dodecyl sulfate polyacrylamide gel electrophoresis (SDS-PAGE), high-performance liquid chromatography (HPLC), ammonium sulfate precipitation, and gel filtration.

When a full-length antibody is desired, coding sequences of any of the V_{H} and V_{L} chains described herein can be linked to the coding sequences of the Fc region of an immunoglobulin and the resultant gene encoding a full-length antibody heavy and light chains can be expressed and assembled in a suitable host cell, e.g., a plant cell, a mammalian cell, a yeast cell, or an insect cell.

Antigen-binding fragments can be prepared via routine methods. For example, F(ab)₂ fragments can be generated by pepsin digestion of an full-length antibody molecule, and Fab fragments that can be made by reducing the disulfide bridges of F(ab′)₂ fragments. Alternatively, such fragments can also be prepared via recombinant technology by expressing the heavy and light chain fragments in suitable host cells and have them assembled to form the desired antigen-binding fragments either in vivo or in vitro. A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

### IV. Chimeric Antigen Receptor (CARs)

A chimeric antigen receptor (CAR) is an artificial immune cell receptor that allow the T cells to recognize a particular antigen of a targeted cell (e.g. a tumor cell). In general, a CAR is a fusion polypeptide comprising an antigen-binding extracellular domain that recognizes a target antigen, a transmembrane domain and an intracellular signaling domain. A T cell that expresses a CAR polypeptide is referred to as a CAR T cell that can specifically target certain antigens of interest.

In some embodiments, a CAR can include a signal peptide at the N-terminus. A signal peptide includes a peptide sequence that directs the transport and localization of the peptide within a cell and/or the cell surface. In one embodiment, the signal peptide includes the signal peptide from human CD8a (MALPVTALLLPLALLLHAARP) (SEQ ID NO: 11). In another embodiment, the signal peptide includes the signal peptide from human CD8b (MRPRLWLLLAAQLTVLHGNSV) (SEQ ID NO: 12). Other examples include the signal peptide from human CD45 and interleukin-2 (IL-2). Functional equivalents are included which are, for example, CD8a signal peptides, CD8b signal peptides, CD45 signal peptides and IL-2 signal peptides from homologous proteins from other species. Signal peptides may cleave either during or after translocation to generate a free signal peptide and a mature protein.

The antigen-binding extracellular domain is the region of a CAR polypeptide exposed to the extracellular fluid when the CAR is expressed on cell surface. Typically, the antigen-binding extracellular domain is a single-chain variable fragment (scFv) derived from a monoclonal antibody while it can be based on other formats which comprise an antibody-like antigen binding site. A scFv may include an antibody heavy chain variable region (V_{H}) and an antibody light chain variable region (V_{L}), having a V_{H}-V_{L} or a V_{L}-V_{H} orientation. In some embodiments, the antigen-binding extracellular domain is a scFv derived from anti-PD-L1 antibodies as described herein e.g. mTT-01. In some embodiments, the V_{H} and V_{L} may be linked to each other via a peptide linker. The peptide linker may be 5-25 amino acid residues in length, 25-100 amino acid residues in length, or 50-200 amino acid residues in length. In some embodiments, the peptide linker is a Gly-Ser linker. In a particular example, a Gly-Ser linker includes the amino acid sequence of GGGGGGGGSGGGGS (SEQ ID NO: 13). In some embodiments, the antigen-binding extracellular domain includes, from N-terminus to C-terminus, mTT-01 V_{H}, a linker and mTT-01 V_{L}. In a particular example, the antigen-binding extracellular domain contains the amino acid sequence set forth in SEQ ID NO: 14. In some embodiments, the antigen-binding extracellular domain includes, from N-terminus to C-terminus, mTT-01 V_{L}, a linker and mTT-01 V_{H}. In a particular example, the antigen-binding extracellular domain contains the amino acid sequence set forth in SEQ ID NO: 15

TABLE 3
Amino acid sequences of antigen-binding
extracellular domains based on mTT-01.

mTT-01 V_{H} + linker + mTT-01 V_{L}
EVMLVESGGALVKPGGSLKLSCAASGFSLSNYVMS
WVRQTPEKRLEWVATISSGGRYIYYTDSVKGRFTI
SRDNARNTLYLQMSSLRSEDTAMYYCARDGSTLYY
FDYWGQGTTLTVSSGGGGSGGGGSGGGGSQTVLTQ
SPAIMSASPGEKVTMTCSASSSVDYMYWYQQKPGS
SPRLLIYDTSNLASGVPVRFSGSGSGTSYSLTISR
MEAEDAATYYCQQWSSSPPITFGTGTKVELK
(SEQ ID NO: 14)
mTT-01 V_{L} + linker + mTT-01 VH
QTVLTQSPAIMSASPGEKVTMTCSASSSVDYMYWY
QQKPGSSPRLLIYDTSNLASGVPVRFSGSGSGTSY
SLTISRMEAEDAATYYCQQWSSSPPITFGTGTKVE
LKGGGGSGGGGSGGGGSEVMLVESGGALVKPGGSL
KLSCAASGFSLSNYVMSWVRQTPEKRLEWVATISS
GGRYIYYTDSVKGRFTISRDNARNTLYLQMSSLRS
EDTAMYYCARDGSTLYYFDYWGQGTTLTVSS
(SEQ ID NO: 15)

The CAR polypeptide described herein may contain a transmembrane domain which is typically an alpha helix comprising several hydrophobic residues that spans the cell membrane. The transmembrane domain can provide stability of the CAR polypeptide containing it. In some embodiments, the transmembrane domain can be a CD28 transmembrane domain, a CD8 transmembrane domain, or a chimera of a CD8 and CD28 transmembrane domain. In some embodiments, the transmembrane domain is a CD8a transmembrane domain containing the sequence of:

(SEQ ID NO: 16)
IWAPLAGTCGVLLLSLVITLYC

The CAR polypeptide described herein may contain a hinge domain located between the transmembrane domain and the antigen binding domain. In some embodiments, a hinge domain may comprise up to 300 amino acids e.g. 5 to 20 amino acids, 15 to 50 amino acids, 20 to 100 amino acids or 30 to 200 amino acids. A hinge domain may provide flexibility to the CAR, or to prevent steric hindrance of the CAR. In some embodiments, the hinge domain is a CD8 hinge domain. In a preferred embodiment, the CD8 hinge domain is human. In some embodiments, the hinge domain is a CD28 hinge domain. In a preferred embodiment, the CD28 hinge domain is human. In some embodiments, the hinge domain is a CD8a hinge domain containing the sequence of:

(SEQ ID NO: 17)
TTTPAPRPPTPAPTIASQPLSLRPEACRPAAGGAVHTRGLDFACDIY.

The intracellular signaling domain of a CAR polypeptide is capable of activating at least one of the normal effector functions of the immune cell engineered to express the CAR polypeptide. Effector function of a T cell, for example, may be cytolytic activity or helper activity including the secretion of cytokines. The intracellular signaling domain of a CAR polypeptide can be a portion of a protein that transduces the effector function signal and directs the cell to perform a specialized function. In particular, the intracellular signaling domain is derived from the intracellular signaling domain of a native receptor. Examples of such native receptors include the zeta (ζ) chain of the T-cell receptor or any of its homologs (e.g. delta, gamma, or epsilon). For example, CD3ζ (CD3-zeta) is the cytoplasmic signaling domain of the CD3 complex of the T cell receptor (TCR). It contains three immunoreceptor tyrosine-based activation motifs (ITAMs) that activate downstream signaling pathways. In some embodiments, the intracellular signaling domain include a CD3-zeta signaling domain containing the sequence of: RVKFSRSADAPAYKQGQNQLYNELNLGRREEYDVLDKRRGRDPEMGGKPRR KNPQEGLYNELQKDKMAEAYSEIGMKGERRRGKGHDGLYQGLSTATKDTYD ALHMQALPPR (SEQ ID NO: 18). CD3-zeta may provide a primary T cell activation signal but not a fully competent activation signal and thus additional co-stimulatory signaling may be needed. A costimulatory molecule is a cell surface molecule other than an antigen receptor or its ligands that is required for an efficient response of lymphocytes to an antigen. Examples of costimulatory molecules include but not limited to CD27, CD28, 4-1BB (CD137), OX40, CD30, lymphocyte function-associated antigen-1 (LFA-1) and CD2. In some instances, the co-stimulatory domains of CD28 and/or 4-1BB may be used to transmit a full proliferative/survival signal, together with the primary signaling mediated by CD3ζ. In some embodiments, a CAR polypeptide disclosed herein comprises a CD28 co-stimulatory molecule. In some embodiments, a CAR polypeptide disclosed herein comprises a 4-1BB co-stimulatory molecule. In some embodiments, the co-stimulatory molecule includes a 4-1BB co-stimulatory molecule containing the sequence of: KRGRKKLLYIFKQPFMRPVQTTQEEDGCSCRFPEEEEGGCEL (SEQ ID NO: 19). In some embodiments, a CAR polypeptide disclosed herein comprises a CD3ζ signaling domain with a CD28 co-stimulatory domain. In some embodiments, a CAR polypeptide disclosed herein comprises a CD3ζ signaling domain with a 4-1BB co-stimulatory domain. In still other embodiments, a CAR includes a CD3ζ signaling domain with a CD28 co-stimulatory domain and a 4-1BB co-stimulatory domain.

In one particular embodiment, a CAR polypeptide disclosed herein comprises the amino acid sequence as set forth in SEQ ID NO: 20 (TT0001: V_{H}-linker-V_{L} chain+Ala-Ala-Ala+CD8a hinge+CD8a transmembrane domain+4-1BB+CD3-zeta). In another particular embodiments, a CAR polypeptide disclosed herein comprises the amino acid sequence as set forth in SEQ ID NO: 21 (TT0002: V_{L}-linker-V_{H} chain+Ala-Ala-Ala+CD8a hinge+CD8a transmembrane domain+4-1BB+CD3-zeta).

TABLE 4
Amino acid sequences of a CAR polypeptide
disclosed herein based on mTT-01.

TT0001: V_{H}-linker-V_{L} chain + Ala-Ala-Ala +
CD8a hinge + CD8a transmembrane domain +
4-1BB + CD3-zeta
EVMLVESGGALVKPGGSLKLSCAASGFSLSNYVMS
WVRQTPEKRLEWVATISSGGRYIYYTDSVKGRFTI
SRDNARNTLYLQMSSLRSEDTAMYYCARDGSTLYY
FDYWGQGTTLTVSSGGGGSGGGGSGGGGSQTVLTQ
SPAIMSASPGEKVTMTCSASSSVDYMYWYQQKPGS
SPRLLIYDTSNLASGVPVRFSGSGSGTSYSLTISR
MEAEDAATYYCQQWSSSPPITFGTGTKVELKAAAT
TTPAPRPPTPAPTIASQPLSLRPEACRPAAGGAVH
TRGLDFACDIYIWAPLAGTCGVLLLSLVITLYCKR
GRKKLLYIFKQPFMRPVQTTQEEDGCSCRFPEEEE
GGCELRVKFSRSADAPAYKQGQNQLYNELNLGRRE
EYDVLDKRRGRDPEMGGKPRRKNPQEGLYNELQKD
KMAEAYSEIGMKGERRRGKGHDGLYQGLSTATKDT
YDALHMQALPPR
(SEQ ID NO: 20)
TT0002: V_{L}-linker-V_{H} chain + Ala-Ala-Ala +
CD8a hinge + CD8a transmembrane domain +
4-1BB + CD3-zeta
QTVLTQSPAIMSASPGEKVTMTCSASSSVDYMYWY
QQKPGSSPRLLIYDTSNLASGVPVRFSGSGSGTSY
SLTISRMEAEDAATYYCQQWSSSPPITFGTGTKVE
LKGGGGSGGGGSGGGGSEVMLVESGGALVKPGGSL
KLSCAASGFSLSNYVMSWVRQTPEKRLEWVATISS
GGRYIYYTDSVKGRFTISRDNARNTLYLQMSSLRS
EDTAMYYCARDGSTLYYFDYWGQGTTLTVSSAAAT
TTPAPRPPTPAPTIASQPLSLRPEACRPAAGGAVH
TRGLDFACDIYIWAPLAGTCGVLLLSLVITLYCKR
GRKKLLYIFKQPFMRPVQTTQEEDGCSCRFPEEEE
GGCELRVKFSRSADAPAYKQGQNQLYNELNLGRRE
EYDVLDKRRGRDPEMGGKPRRKNPQEGLYNELQKD
KMAEAYSEIGMKGERRRGKGHDGLYQGLSTATKDT
YDALHMQALPPR
(SEQ ID NO: 21)

### V. Nucleic Acid (Vector) and CAR Expressing Cell

A nucleic acid can be provided which encodes a CAR as described herein. The nucleic acid sequence may be, for example, a DNA, an RNA or a cDNA sequence. A nucleic acid encoding a CAR can be inserted into a vector. The vector may be a plasmid or a viral vector. The vector may be capable of transfecting or transducing a T cell. For example, a viral vector can be used such as a retrovirus vector (e.g. an oncoretrovirus vector, a lentivirus vector, and a pseudotyped vector), an adenovirus vector, an adeno-associated virus (AAV) vector, a simian virus vector, a vaccinia virus vector or a sendai virus vector, an Epstein-Barr virus (EBV) vector, and a herpes simplex virus (HSV) vector.

A nucleic acid encoding a CAR can be introduced into a cell. In some embodiments, a nucleic acid encoding a CAR can be designed to insert into a genomic site of interest in the host T cells. In some embodiments, the target genomic site can be in a safe harbor locus. Specifically, a nucleic acid encoding a CAR can insert into a location within a TRAC gene to disrupt the TRAC gene in the genetically engineered T cells and express the CAR polypeptide. Disruption of TRAC results in loss of function of the endogenous TCR. A viral vector such as an AAV vector or a LV vector, which encodes a CAR polypeptide described herein may be incubated with T cells for a suitable period to allow for entry of the viral vector into the T cells. After transduction, the T cells may be cultured in a suitable cell culture medium for a suitable period for recovery. The genetically engineered T cells may be expanded in vitro under suitable conditions to produce a population of genetically engineered T cells as desired.

The cell used as in the genetic engineering process for expressing a CAR is not particularly limited. The cell may be any eukaryotic cell capable of expressing a CAR at the cell surface, e.g. an immunological cell. In particular, the cell may be an immune effector cell such as a T cell. The T cell may include helper T cells (TH cells), cytotoxicity T cells (CTLs), memory T cells and regulatory T cells (Treg cells). The cells may be from a sample isolated from a patient, a related or unrelated haematopoietic transplant donor or a completely unconnected donor, from cord blood, differentiated from an embryonic cell line or an inducible progenitor cell line, or derived from a transformed cell line. In some embodiments, CAR expressing cells may be created from a peripheral blood mononuclear cell (PBMC) which may be obtained from a patient's own peripheral blood, or a haematopoietic stem cell transplant from donor peripheral blood. The CAR expressing cells as produced may be further expanded in vitro under suitable conditions to produce a population of CAR expressing cells to an amount as needed e.g. a clinically relevant scale. The CAR expressing cells as produced as described herein may be harvested for therapeutic uses using

### VI. Pharmaceutical Composition

The present invention also relates to a pharmaceutical composition comprising a CAR expressing cell as described herein. The CAR expressing cell may be formulated with a pharmaceutically acceptable carrier for purpose of delivery.

As used herein, “pharmaceutically acceptable” means that the carrier is compatible with an active ingredient in the composition, and preferably can stabilize said active ingredient and is safe to the receiving individual. Said carrier may be a diluent, vehicle, excipient, or matrix to the active ingredient. Typically, a composition comprising a CAR expressing cell as described herein as an active ingredient can be in a form of a solution such as an aqueous solution e.g. a saline solution. Appropriate excipients also include lactose, sucrose, dextrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may further contain pharmaceutically acceptable auxiliary substances as required to approximate physiological conditions, for example, pH adjusting and buffering agents, such as sodium acetate, sodium chloride, potassium chloride, calcium chloride, sodium lactate and the like. The composition of the present invention may be delivered via a physiologically acceptable route, typically intravenous infusion.

### V. Treatment

A population of genetically engineered CAR expressing cells as described herein may be administered to a subject for therapeutic purpose. In particular, the present invention provides a method for treating a disease a subject having a disease, disorder or condition associated with an elevated expression of a tumor antigen comprising administering to the subject an effective amount of a cell genetically modified to express a CAR as described herein. The present invention also provides use of a cell genetically modified to express a CAR as described herein for manufacturing a medicament for treating a disease a subject having a disease, disorder or condition associated with an elevated expression of a tumor antigen.

The term “effective amount” used herein refers to the amount of an active ingredient to confer a desired biological effect in a treated subject. The effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience. In some examples, the effective amount of an active ingredient is to provide an anti-tumor effect such as reducing tumor size as compared with that without administration of the active ingredient. In some embodiments, an effective amount of a genetically engineered cell population may comprise 10⁵ to 10⁷ cells, such as 1×10⁵ cells, 2×10⁵ cells, 3×10⁵ cells, 4×10⁵ cells, 5×10⁵ cells, 6×10⁵ cells, 7×10⁵ cells, 8×10⁵ cells, 9×10⁵ cells, 1×10⁶ cells, 2×10⁶ cells, 3×10⁶ cells, 4×10⁶ cells, 5×10⁶ cells, 6×10⁶ cells, 7×10⁶ cells, 8×10⁶ cells, 9×10⁶ cells, 1×10⁷ cells, 2×10⁷ cells, 3×10⁷ cells, 4×10⁷ cells, 5×10⁷ cells, 6×10⁷ cells, 7×10⁷ cells, 8×10⁷ cells, 9×10⁷ cells, or multiples thereof.

A subject to be treated by the method of treatment as described herein can be a mammal, more preferably a human. Mammals include, but are not limited to, farm animals, sport animals, pets, primates, horses, dogs, cats, mice, and rats. A human subject who needs the treatment may be a human patient having, at risk for, or suspected of having a target disease/disorder. A subject suspected of having any of such target disease/disorder might show one or more symptoms of the disease/disorder. A subject at risk for the disease/disorder can be a subject having one or more of the risk factors for that disease/disorder.

In some embodiments, the subject has been determined to have a relatively higher level of a tumor antigen as compared to a reference level.

In some embodiments, the method may include measuring a tumor antigen in a tumor sample from a patient and comparing the level of the tumor antigen in the sample with a reference level. In some embodiments, based on the comparing, a patient determined to have an enhanced level of the tumor antigen is selected. Specifically, for example, an enhanced level can be higher than a reference level by more than 5%, 10%, 20%, 30%, 40%, 50%, 60%, 70%, 80%, 90% or 100% or more. A reference level with respect to a tumor antigen as described herein can refer to a level measured in control samples (e.g. tissues or cells or any biological sample free of cancer of an individual or from a population of normal individuals). The measurement can be performed using conventional detection and statistic methods.

In some embodiments, the tumor antigen is PD-L1.

In some embodiments, the subject is suffered from cancer. Non-limiting examples of cancers that may be treated using a population of genetically engineered described herein include, but are not limited to, breast cancer, colon cancer, head and neck cancer, thyroid cancer, lung cancer, ovarian cancer, pancreatic cancer, gastric cancer, kidney cancer, glioblastoma and leukemia.

Administering may include transplantation of the genetically engineered CAR expressing cells by a method or route such that a desired amount of the genetically engineered CAR expressing cells delivered to and located at a desired site, such as a tumor site, leading to a desired therapeutic effect(s). For example, in some instances, an effective amount of the genetically engineered CAR expressing cells can be administered via a systemic route of administration, such as injection and infusion. Injection includes, without limitation, intravenous, intramuscular, intra-arterial, intrathecal, intracardiac, intraperitoneal, subcutaneous. In some particular embodiments, the route is intravenous.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### 1. Material and Methods

### 1.1 Anti-PD-L1 Monoclonal Antibody

Anti-PD-L1 monoclonal antibody mTT-01 was prepared as described in U.S. Pat. No. 11,660,352 B2 which has a V_{H} domain of SEQ ID NO: 7 and a V_{L} domain of SEQ ID NO: 8, as provided in Table 1. Chimeric-atezolizumab (anti-hPD-L1-mIgG1) was purchased from InvivoGen (Anti-hPD-L1-mIgG1 InvivoFit, Cat #: hpdl1-mab9-1), which is a recombinant monoclonal antibody that features the constant region of a mouse IgG1 isotype and the variable region of Atezolizumab.

### 1.2 Immunohistochemistry Assay for Human Tissues

For the immunohistochemical analysis, the following modified protocol was utilized. Slides, containing either formalin-fixed paraffin-embedded (FFPE) tissue or tissue microarrays (TMAs) sourced from SCMH, were initially prepared by incubating at 60-65° C. (for FFPE tissue) or 70° C. (for TMAs) for 30 minutes. Deparaffinization was achieved through two consecutive 10-minute immersions in Surgipath Xylene (3803665, Leica). The slides were then rehydrated through a graded series of Surgipath Reagent Alcohol 100% (3803686, Leica), decreasing in concentration from 100%, 95%, 80%, to 70%, each step lasting 5 minutes, followed by a rinse in tap water.

Antigen retrieval was performed using Citrate-based Antigen Retrieval Solution, pH 6 (S2369, DAKO), suitable for mTT-01 antigen, in a pressure cooker for 30 minutes. After cooling to room temperature, sections were washed in Tris-buffered saline with Tween 20 (TBST; TBT999, Scytek) for 5 minutes. Endogenous peroxidase activity was blocked by incubating sections in Hydrogen Peroxide Blocking Solution (TA-060-HP, Thermo Fisher Scientific) for 10 minutes, followed by two 3-minute washes in TBST. Non-specific binding was blocked using 5% Normal Goat Serum (005-000-001, Jackson ImmunoResearch Laboratories, Inc.) for 30 minutes, followed by a 10-minute incubation with Protein Blocking Solution (TA-060-PBQ, Thermo Fisher Scientific).

Primary antibody incubation was conducted overnight at 4° C. with 200× diluted mTT-01 antibody, shielded from light. Post-incubation, sections were washed three times in TBST for 3 minutes each. Amplification of primary antibody binding was performed using Primary Antibody Amplifier Quanto (TL-060-QPB) for 10 minutes, followed by three more TBST washes. Detection was achieved using HRP Polymer Quanto (TL-060-QPH) for 10 minutes, and additional three TBST washes.

Visualization of antigen-antibody complexes was performed using DAB Substrate Kits (TA-060-QHSX and TA-002-QHCX, Thermo Fisher Scientific) for 5 minutes. Slides were then rinsed in tap water for 5 minutes, counterstained with Surgipath Hematoxylin Gill II (3801522, Leica) for 2 minutes, and washed again in tap water for 5 minutes. Dehydration was performed through two 5-minute immersions in 100% alcohol, followed by two 5-minute clearings in Xylene. The slides were then mounted using Surgipath Micromount Mounting Medium (3801731. Leica).

### 1.3 CAR Constructs and Production of CAR T-Cells

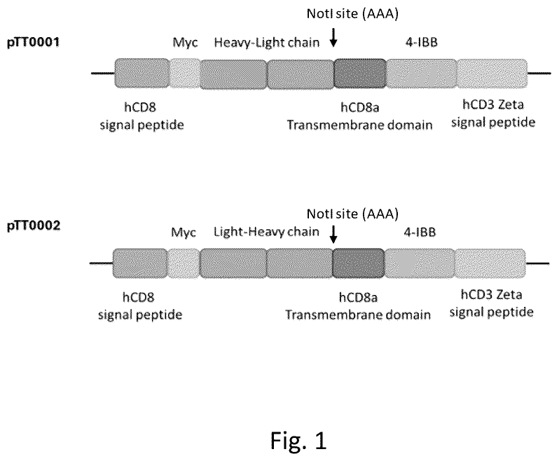

CARs were constructed and expressed based on the anti-PD-L1 monoclonal antibody mTT-01. Specifically, the gPD-L1 specific second-generation CARs were designed to harbor scFv, obtained from mTT-01 and linked in the order of V_{H} to V_{L} (pTT0001) or V_{L} to V_{H} (pTT0002), followed by a hCD8 hinge, transmembrane domain, 4-1BB co-stimulatory domain, and CD3ζ signaling domain. The scFv sequences were obtained from the mTT-01 monoclonal antibody. The structure of the CAR constructs is shown in FIG. 1. The CAR constructs were incorporated into a lentiviral vector using In-Fusion cloning (Takara Bio, Shiga-ken, Japan). Primary human CD3+ T cells were isolated from the PBMC of healthy donors using CD3 MicroBeads (Miltenyi Biotec, North Rhine-Westphalia, Germany) and stimulated by ImmunoCult™ Human CD3/CD28/CD2 T Cell Activator (Stem Cell Technologies, Vancouver, Canada) with 100 IU/ml of Proleukin (Novartis, Basel, Swiss). After 3 days, the activated CD3+ T cells were transduced with RetroNectin (Takara Bio, Shiga-ken, Japan). Lentivirus containing supernatant was removed at two days post-infection. The culture medium of CAR T-cells was replaced every 2-3 days with fresh Proleukin-containing media. The levels of CAR expression on T cells were measured at 7 days after transduction using flow cytometry. In vitro Cytotoxicity assay and Xenograft mouse model were performed with CAR T-cells at day 14.

### 1.4 Cell Culture

Breast cancer cell lines (MDA-MB-231, MDA-MB-468, BT549, BT474 and MCF-7), human non-small cell lung cancer (NSCLC) A549 cells and 293T cells were purchased from ATCC. Jurkat-Lucia™ NFAT Cells was purchased from InvivoGen (InvivoGen, CA, USA). MDA-MB-231 PD-L1 cells were established by overexpression of human PD-L1 using a lentivirus system as previously described¹⁰, and MDA-MB-231 PD-L1^{KO} cells. BT474 and Jurkat-Lucia™ NFAT cells were cultured in RPMI 1640 (Gibco, Gaithersburg, MD) containing 10% heat-inactivated fetal bovine serum (Cytiva, MA, USA) and 1% Penicillin-Streptomycin (Gibco, Gaithersburg, MD). Other cell lines were maintained in DMEM/F12 (Gibco, Gaithersburg, MD) supplemented with 10% heat-inactivated fetal bovine serum and 1% Penicillin-Streptomycin. All cell lines were incubated in a humidified incubator with 5% CO₂ at 37° C.

### 1.5 Luciferase Assay

TT0001 and TT0002 Jurkat NFAT reporter luciferase (Luc) cell line was generated by transduction of Jurkat-Lucia™ NFAT cells with the TT0001 and TT0002 lentivirus. The stable expression of CARs on TT0001 and TT0002 Jurkat NFAT-Luc cell surfaces was confirmed by flow cytometry. MDA-MB-231, BT549, A549 and 293T (1×10⁵ cells/well) were co-incubated with TT0001 and TT0002 Jurkat NFAT-Luc cells for two days at a 1:1 effector-to-target (E:T) ratio. QUANTI-Luc™ (InvivoGen, CA, USA) was used to monitor luciferase released from TT0001 and TT0002 Jurkat NFAT-Luc cells after activation by the PD-L1 expressing cells. The luminescence (RLU) was measured using GloMax 96 Microplate Luminometer (Promaga, WI, USA) and normalized with unstimulated control.

### 1.6 In Vitro Cytotoxicity Assay

The cytotoxic activity of CAR-T cells was measured using the Cell Counting Kit-8 (CCK-8) assay (Dojindo, MD, USA). MDA-MB-231, MDA-231 PD-L1, MDA-MB-231 PD-L1^{KO} cells (3×10⁴ cells/well) were co-cultured with CAR T-cells for 24 at the E:T ratio of 10:1, 3:1, 1:1, and 0.3:1. MCF-7, BT549, M10, BT474, ZR75-1, and MDA-MB-361 cells (1.5×10⁴ cells/well) were co-cultured with CAR T-cells for 24 hr at the E:T ratio of 3:1. CAR T-cells were then removed from the target cells by washing with PBS. The viability of the tumor cells was evaluated using CCK-8 and lysis activity was calculated according to the following formula: % lysis=1−(CCK-8 ratio from CAR T-cells treated wells)/(CCK-8 ratio from untreated wells)×100%.

### 1.7 Multi-Plex Immunoassay Analysis

Supernatants were collected from co-incubation of CAR T-cells with MDA-MB-231, MDA-231 PD-L1 or MDA-MB-231 PD-L1^{KO} at various E:T ratios for 48 h and centrifuged at 300×g for 10 min to remove cell debris. The levels of human TNF-α, IL-6, IFN-γ, IL-10, and IL-2 release were measured by Multi-Plex Immunoassay (MPI) performed by the Inflammation Core Facility (Institute of Biomedical Sciences, Academia Sinica, Taiwan, supported by AS-CFII-108-118). Antibody conjugated magnetic beads were incubated with cytokine-containing samples, washed, probed with biotinylated antibodies, and subsequently labeled with Streptavidin-Phycoerythrin (PE). The fluorescence levels of the beads were detected by the Bio-Plex® 200 system (Bio-Rad, CA, USA) and the concentrations of the cytokines were normalized with the manufacturer's provided standards. All samples in this assay were protected from light and performed at room temperature.

### 1.8 Fluorescence-Activated Cell Sorting (FACS) Binding Assay

To evaluate the expression levels of gPD-L1 in cancer cell lines, cells were incubated with 1 μg of mTT-01 monoclonal antibody (Topmunnity Therapeutics, Taiwan) in FACS buffer (2% FBS in PBS) at 4° C. for 30 min. The cells were then washed with FACS buffer and incubated with 0.25 μg of Alexa Fluor® 647 Goat Anti-Human IgG secondary antibody in FACS buffer for 30 minutes at 4° C. 7-amino-actinomycin D (7-AAD) staining was used to distinguish viable cells before analysis. The transduction efficiency and the subset of CAR T-cells were measured using flow cytometry.¹¹. Briefly, CAR T-cells were collected and blocked with Human TruStain FcX (Biolegend, CA, USA) in FACS buffer at 4° C. for 15 min, followed by staining with Anti-c-myc antibody (Roche, Basel, Switzerland) at 4° C. for 30 min. After washing, the cells were incubated with Alexa Fluor® 488 Goat Anti-Mouse IgG secondary antibody (Thermo Fisher, MA, USA) in FACS buffer at 4° C. for 30 min. After that, cells were probed with monoclonal antibodies against human CD3, CD4, CD8, CD45RA, CD45RO, and CD62L (Biolegend, CA, USA) at 4° C. for 30 min. The live cells were determined using eBioscience™ Fixable Viability Dye eFluor™ 780 (Thermo Fisher, MA, USA). The stained cells were analyzed by an Attune NXT Cytometer (Thermo Fisher, MA, USA), and the data was organized using FlowJo V10 software. To determine the memory function of CAR T-cells, blood, spleen, and Bone marrow were collected from CAR T-cells treated mice after tumor disappear. The staining procedures and antibody panels were followed by the condition in transduction efficiency and the subset of CAR T-cells.

### 1.9 Xenograft Mouse Model

All animal experiment protocols were reviewed and approved by the IACUC. Six-week-old female NOD.Cg-Prkdc_{scid} Il2rg^{tm1Wjl}/SzJ (NSG) mice were purchased from and maintained in Animal Core Facility (Institute of Biomedical Sciences, Academia Sinica, Taiwan). To establish a breast cancer tumor model, mice were subcutaneously inoculated with 5×10⁶ MDA-MB-231 or BT549 cells (triple negative breast cancer) pre-mixed with Matrigel Matrix (Corning, NY, USA) at a 1:1 ratio. Mice were received with 2 doses of CAR T-cells (total of 2×10⁷ cells in the MDA-MB-231 model or 1×10⁷ cells in the BT549 model) after tumor size growth above 100 mm³. To assess the memory phenotype of CAR T-cells, mice were rechallenged with 1×10⁷ MDA-MB-231 or BT549 cells once the tumor disappeared for 12 days in a row. The sizes of tumors were calculated using the formula: V=(length×width²)/2.

### 1.10 Immunohistochemistry and Immunofluorescence Assay for Mouse Tissues

Intracardial perfusion of mice prior to tumor tissue extraction was performed to assess the infiltration of CAR T-cells. Tumor tissues were processed as previously described, with some modifications^{11 12}. Formalin-fixed paraffin embedded (FFPE) tumor sections were deparaffinized with xylene, rehydrated in graded ethanol, and rinsed with distilled water. Antigen retrieval was performed with 1× Citrate Buffer (C9999, Sigma) at 95° C. for 30 min. After cooling to RT and PBST (PBS+0.05% Tween 20) washing for three times, tumor sections were blocked with 2% donkey serum in PBS with 1% BSA and 0.5% triton for 30 min at RT, and then incubated with primary antibodies against hPD-L1 (1:1000) (ab205921, Abcam, Cambridge, UK) hCD3 (1:100) (MCA1477, Bio-Rad, CA, USA), hCD8a (1:50) (ab17147, Abcam, Cambridge, UK) and human Granzyme B (1:70) (ab4059, Abcam, Cambridge, UK) at 4° C. overnight. For immunohistochemistry, tissues were washed three times with 1×PBST and incubated with ImmPRESS® horse anti-rabbit, mouse, rat IgG polymer kit (MP-5401, MP-5404, MP-5402 and Vector Laboratories, Burlingame, CA, USA) for 30 min at RT. Sections were then detected with ImmPACT® Vector® Red (SK-5105, Vector Laboratories). Final slides were counterstained in hematoxylin. Images were scanned by PANNORAMIC 250 Flash III (3DHISTECH, Budapest, Öv u. 3, Hungary) For immunofluorescence assay, tissues were washed three times and incubated for 1 hour at RT with secondary antibodies Alexa Fluor® 488 Goat Anti-Rat IgG (1:500), Alexa Fluor® 568 Anti-Mouse IgG (1:1000), and Alexa Fluor® 647 Donkey Anti-Rabbit (1:1000). Nuclei were stained with 4′,6-diamidino-2-phenylindole (DAPI) in the dark for 10 min. Images were taken by a LSM 700 stage confocal laser scanning microscope (Zeiss, Oberkochen, Germany).

### 1.11 Biodistribution

To detect the distribution of CAR T-cells, seven female NSG mice were inoculated with MDA-MB-231 cells (1×10⁷ cells) prior to intravenous injection of CAR T-cells (1×10⁷ cells). Blood, lung, spleen, liver, kidney, mesenteric lymph node, brain, and tumor tissues were collected from the mice at 4 h, D1, D2, D3, D7, D14, and D21 post-inoculation. Genomic DNA (gDNA) was extracted from these tissues by QIAamp DNA Mini Kit (Qiagen, Hilden, Germany) according to the manufacturer's instruction. The concentrations of gDNA were measured using Nanodrop2000 (Thermo Fisher, MA, USA) and adjusted to a suitable concentration range. The copy numbers of the TT0002 gene were quantified using qPCR on CFX connect real-time PCR detection system (Bio-Rad, France). Primers for TT0002 and mouse β-actin are listed in Table 5 (TT0002-4 and mus b-actin).

TABLE 5
Primers for TT0002 and mouse ß-actin
(TT0002-4 and mus beta-actin)

| Primers | Sequences (5′ to 3′) |
|---|---|
| TT0002-4 F | CTG GGA CCT CTT ACT CTC |
| | (SEQ ID NO: 22) |
| TT0002-4 R | CCA GTA CCG AAC GTG ATG |
| | (SEQ ID NO: 23) |
| mus beta- | TGT CAC TCT TCT CTT AGG TAT GGA |
| actin F | (SEQ ID NO: 24) |
| mus beta- | GGT CTT TAC GGA TGT CAA CG |
| actin R | (SEQ ID NO: 25) |

The qPCR protocols consisted of 95° C. for 3 min, followed by 40 cycles of 95° C. for 10 s and 60° C. for 30 s. Each of the primer specificities was confirmed by melting curves following the reaction.

### 1.12 Statistical Analysis

Two independent groups were analyzed using Student's t-test, and multiple groups was performed using one-way ANOVA. The data was presented as mean±SD (standard deviation) and p-values ≤0.05 were considered statistically significant. All statistical analyses were performed using GraphPad Prism v8.0 (GraphPad Software, San Diego, CA).

### 2. Results

### 2.1 Immunohistochemistry Assay

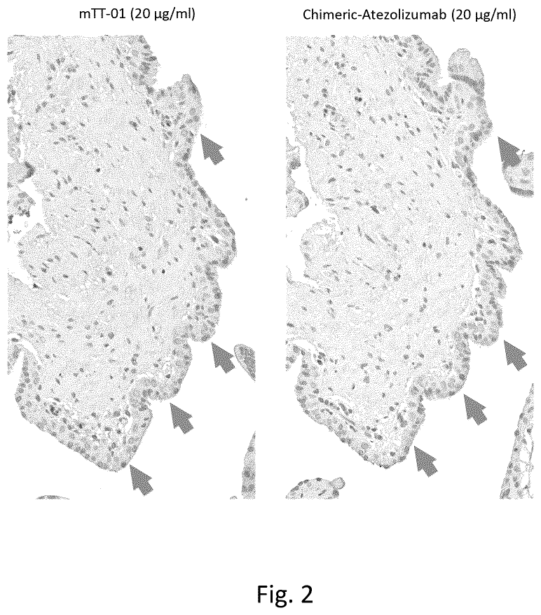

The comparative analysis of binding properties between chimeric-atezolizumab and mTT-01 in human tissues highlights the enhanced specificity of mTT-01, attribute to its interaction with different N-glycosylated sites on PD-L1 compared to those targeted by chimeric-atezolizumab (FIGS. 2-5).

Human placenta, tonsil and breast cancer tissue were used to compare the binding properties of mTT-01 to chimeric-atezolizumab (Anti-hPD-L1-mIgG1 InvivoFit, Cat #: hpdl1-mab9-1, InvivoGen). Chimeric-atezolizumab (Anti-hPD-L1-mIgG1) features the constant region of the mouse IgG1 isotype and the variable region of Atezolizumab. Under the same working concentration (20 μg/ml), positive membrane staining was observed among both mTT-01- and chimeric-atezolizumab-stained placenta tissue (FIG. 2, arrow-pointed). However, the staining intensity was lower in mTT-01-stained tissue (FIG. 2, left panel).

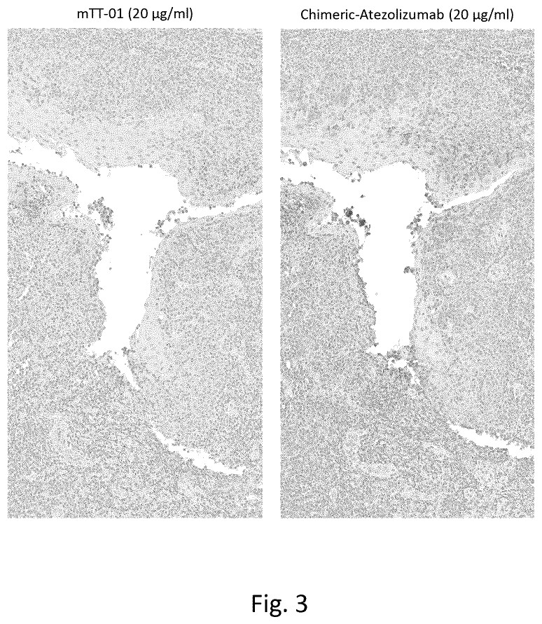

In tonsil tissue, mTT-01 and chimeric-atezolizumab showed similar staining patterns. However, the staining intensity was lower in mTT-01-stained tissue (FIG. 3, left panel).

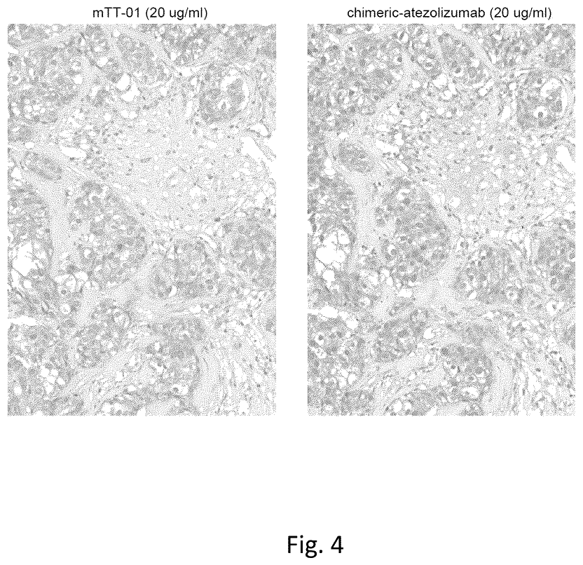

In breast cancer tissue, both mTT-01 and chimeric-atezolizumab showed membrane staining patterns in tumor cells. However, the intensity of cytoplasmic staining was higher in the chimeric-atezolizumab-stained tissue (FIG. 4). Also, background staining in stroma and nucleus was observed in the chimeric-atezolizumab-stained tissue (Table 6).

TABLE 6
Binding Properties Comparisons Between mTT-01
and Chimeric Atezolizumab in Human Placenta,
Tonsil, and Breast Cancer Tissue.

| | | |
|---|---|---|
| | | Chimeric |
| (20 μg/ml) | mTT-01 | Atezolizumab |

Breast cancer tissue

| | | | |
|---|---|---|---|
| Tumor | Membrane staining | +++ | ++ |
| cells | Cytoplasmic | ++ | +++ |
| | staining | | |

| | | |
|---|---|---|
| Immune cells | − | + |
| Stroma | − | ++ |
| Nucleus | − | ++ |

Normal tissue

| | | |
|---|---|---|
| Tonsil | + | ++ |
| Placenta | + | ++ |

The binding property comparison between mTT-01 and chimeric-atezolizumab was further investigated using SCMH breast cancer TMA (BRCA-23). A total of 43 breast cancer cores and 44 cancer adjacent normal tissue cores were evaluated. Detailed qualitative and quantitative analysis of each core can be found in the Topmunnity Database. NO patient demographic information for this TMA. Binding properties of the antibodies are determined by using TPS. The specimen was considered antibody-stained positively if TPS≥50% of the viable tumor cells exhibit membrane staining at any intensity. Binding properties of the antibodies to normal ducts is determined by the same standard that ≥50% of viable ducts show membrane staining at any intensity is defined as positive ductal staining.

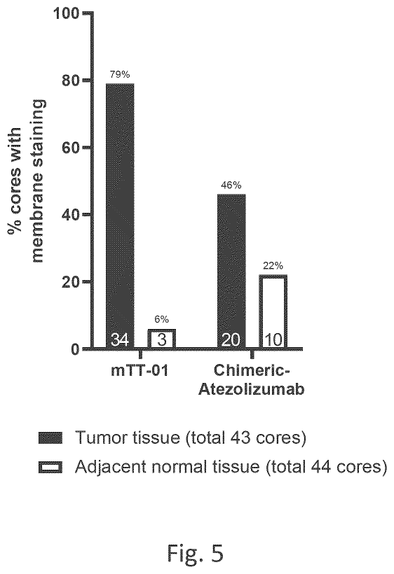

Overall, positive membranous mTT-01 staining was found in 34 out of 43 breast cancer cores (79%, FIG. 5). On the other hand, positive membranous chimeric-atezolizumab staining was found in 20 out of 43 breast cancer cores (46.5%). All (20) breast cancer cores stained positively by chimeric-atezolizumab were also stained positively by mTT-01.

Among 43 breast cancer cores, 26 of them are identified as DCIS core, and the other 17 are invasive carcinoma tissue. Positive membranous mTT-01 staining was found in 22 out of 26 DCIS cores (84.6%), while positive membranous chimeric-atezolizumab staining was found in 17 out of 26 DCIS cores (65.39%). The major difference was found in the positive membranous staining in the invasive carcinoma cores while positive membranous mTT-01 staining was found in 70.6% of cores, only 17.6% of the cores were chimeric-atezolizumab positive.

In cancer adjacent normal tissue, on the contrary, 3 out of 44 cores (3.6%) showed positive membranous mTT-01 staining on normal ducts, while 10 out of 44 cores (22.7%) showed positive membranous chimeric-atezolizumab staining. All (3) adjacent normal cores stained positively by mTT-01 were also stained positively by chimeric-atezolizumab.

### 2.2 Effective CAR Expression in Jurkat Cells

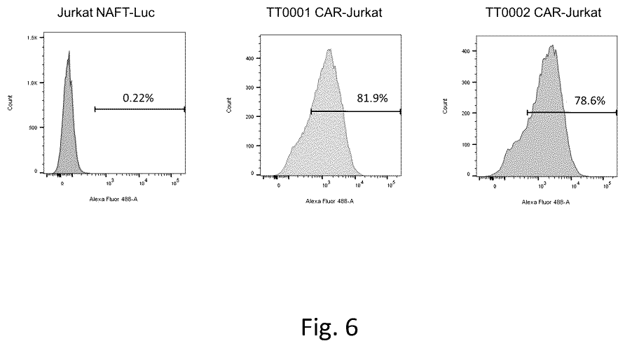

The results, as illustrated in FIG. 6, demonstrate successful integration and expression of Chimeric Antigen Receptors (CARs) in Jurkat NFAT-Luc cells, a T lymphocyte-derived immortal cell line. These cells have been engineered to include an NFAT (Nuclear Factor of Activated T-Cells)-inducible Lucia reporter, which expresses luciferase upon activation of the NFAT pathway. This is crucial in evaluating the functionality of CAR constructs.

In the experiments, TT0001 and TT0002 CAR constructs were transduced into Jurkat NFAT-Luc cells using lentiviral vectors. Subsequent analysis showed detectable expression of myc on the surface of these cells, a marker for CAR presence. In FIG. 6, the left image represents normal Jurkat NFAT-Luc cells, establishing a baseline for myc expression. The middle and right images show the distribution of myc in TT0001 and TT0002 CAR-Jurkat cells, respectively, indicating that 81.9% of TT0001 and 78.6% of TT0002 CAR-Jurkat cells express myc, confirming effective CAR integration and expression.

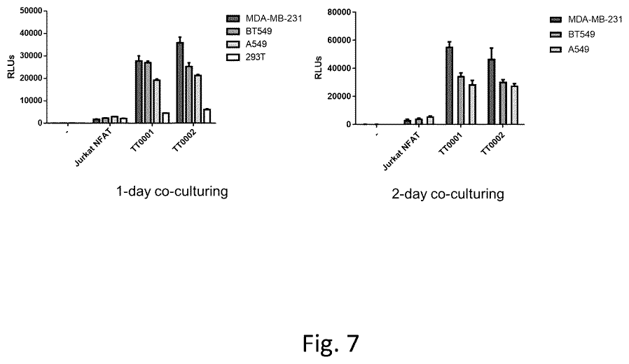

Further bioluminescence intensity of these CAR-modified Jurkat cells is presented in FIG. 7, where CAR-Jurkat cells were co-cultured with various cell lines, including MDA-MB-231, BT549, A549, and HEK293T, to assess their functional response. The bioluminescence intensity from the CAR-Jurkat cells, indicative of NFAT pathway activation, showed a positive correlation with PD-L1 expression on the surface of co-cultured cells. This observation confirms that CAR-Jurkat cells can appropriately activate the NFAT pathway when co-cultured with target cancer cells, demonstrating the functional efficacy of the CAR's signal peptide.

### 2.3 Establishment of CAR Constructs in CD3+ T Cells

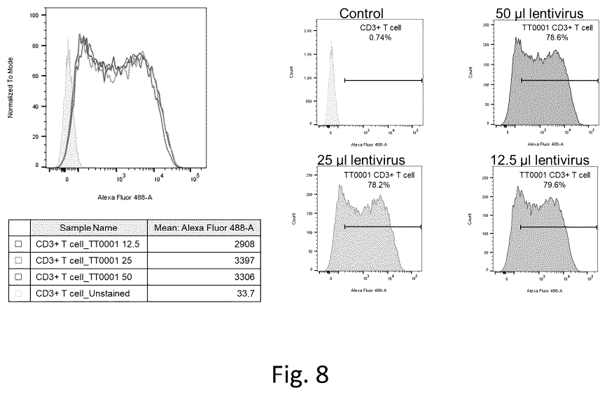

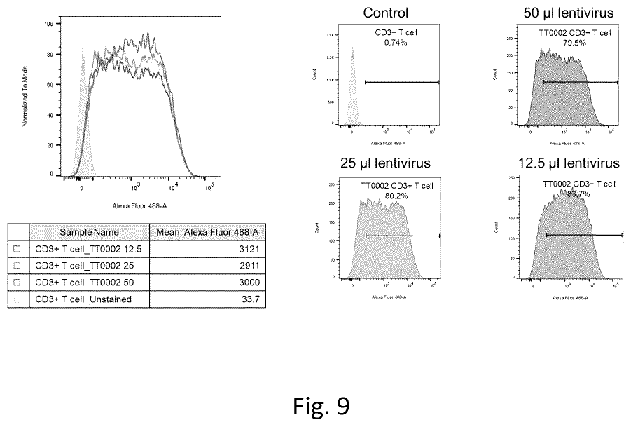

This section of the patent application elaborates on the successful establishment of CAR constructs in primary human CD3+ T cells, as substantiated by FIGS. 8-9. The process involved the infection of these T cells with a lentiviral vector carrying the TT0001 and TT0002 CAR constructs.

For TT0001 CAR-T, as depicted in FIG. 8, the primary CD3+ T cells were transduced with the lentiviral vector containing the TT0001 CAR construct. Post-infection, the surface expression of myc, a marker indicative of CAR expression, was detected using Fluorescence-Activated Cell Sorting (FACS) analysis. The FACS data showed that a significant portion of the T cells, over 78%, expressed myc on their surface with a Mean Fluorescence Intensity (MFI) of approximately 3000. This expression level was notably higher than the normal range of myc expression in untransduced CD3+ T cells, which was determined to be at an MFI of 33.7. This substantial increase in MFI indicates the successful expression of the TT0001 CAR construct in the CD3+ T cells.

Similarly, FIG. 9 focuses on the establishment of the TT0002 CAR-T. The procedure paralleled that of TT0001, where primary CD3+ T cells were transduced with a lentiviral vector encoding the TT0002 CAR construct. The subsequent FACS analysis revealed that over 79% of these transduced T cells expressed myc on their surface with an MFI of around 3000, a significant elevation compared to the baseline expression of myc in normal CD3+ T cells.

The results collectively demonstrate the successful transduction and expression of both TT0001 and TT0002 CAR constructs in CD3+ T cells. This is a pivotal step in the development of CAR-T cell therapies, as it showcases the ability to reprogram primary T cells to express specific CAR constructs, potentially directing them against target antigens for therapeutic applications in cancer treatment.

2.4 Cytotoxicity and PD-L1 Expression Analysis in Breast Cancer Cell Lines with CAR-T Treatment

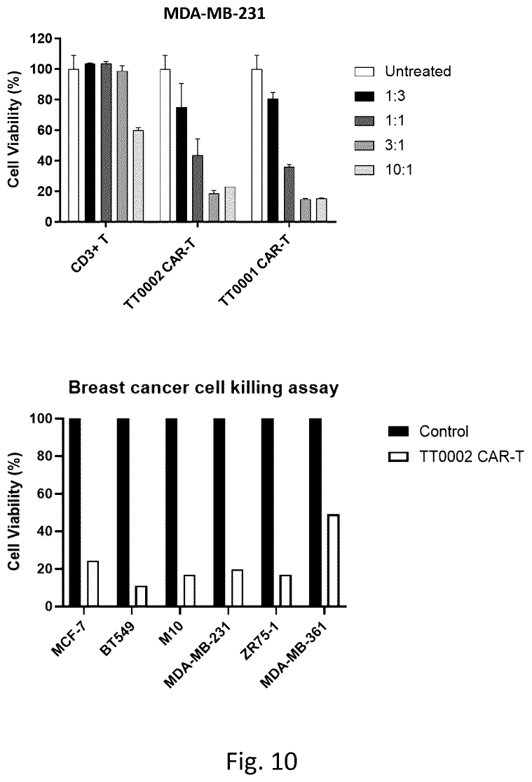

In an extensive evaluation of CAR-T cells against various breast cancer cell lines, a crucial correlation was established between the cytotoxicity of CAR-T cells and the PD-L1 expression levels in these cells, as referenced in FIGS. 10-12. FIG. 10 top panel shows both TT0001 and TT0002 CAR-T exhibiting cytotoxicity function in killing MDA-MB-231 with different E:T ratios. Moreover, FIG. 10 bottom panel revealed that TT0002 CAR-T cells exhibited diverse levels of cytotoxicity across different cell lines, with moderate effects observed in the MDA-MB-361 cell line, negligible impact on the BT474 cell line, and high cytotoxicity in other lines.

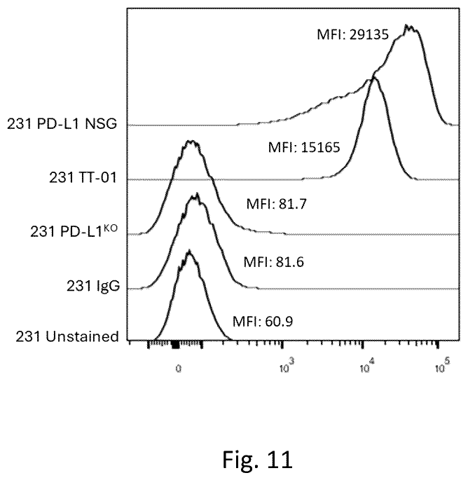

Further delineation of PD-L1 expression in MDA-MB-231 cell lines, as shown in FIG. 11, allowed for a deeper understanding of this variability in response. The results indicated that the cytotoxic efficacy of TT0002 CAR-T cells was not uniform but dependent on the PD-L1 expression levels of the target cells. The MDA-MB-231 PD-L1 overexpressing line and the regular high PD-L1 expressing MDA-MB-231 line showed significant sensitivity to the treatment. In contrast, the PD-L1 knockout variant demonstrated a lack of PD-L1 expression, offering insights into the mechanism of action of TT0002 CAR-T cells.

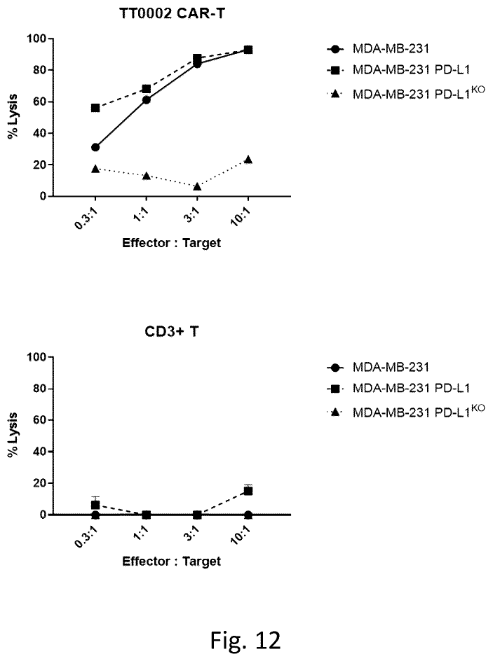

FIG. 12's comparative analysis of TT0001 and TT0002 CAR-T cells further reinforced these observations. It was noted that the cytotoxic effects of both CAR-T cell types correlated positively with the PD-L1 expression levels in the target cells, highlighting the critical role of PD-L1 in modulating the response to CAR-T cell therapy.

These findings underscore the potential of CAR-T cells in treating breast cancer subtypes with varying PD-L1 expressions, offering valuable insights for future therapeutic strategies.

### 2.5 Multi-Plex Analysis Demonstrates Cytokine Production

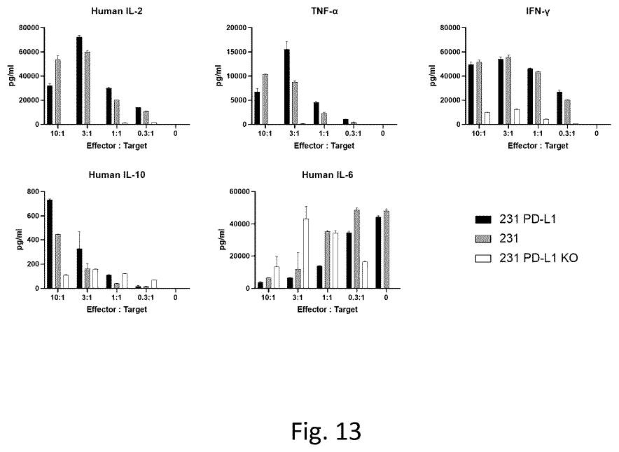

The results from a Multi-Plex assay, as detailed in FIG. 13, demonstrate the efficacy of TT0002 CAR-T cells in in vitro settings. This assay was strategically employed to evaluate the cytokine release profile of these engineered T cells when co-cultured with MDA-MB-231 cell lines that exhibit varying levels of PD-L1 expression (FIG. 11). The assay focused on measuring key cytokines, integral to the immune response and indicative of CAR-T cell activity.

In the upper-left graph of FIG. 13, the IL-2 cytokine measurements revealed that the highest secretion levels occurred in co-cultures with an E:T ratio of 3:1, especially in cell lines expressing PD-L1. This pattern indicates that IL-2 production, a crucial cytokine for T cell activation and proliferation, is augmented in the presence of target cells with PD-L1 expression, validating the responsiveness of CAR-T cells to their intended targets.

The assay also measured TNF-α and IFN-γ, as shown in the middle-upper and upper-right graphs of FIG. 13. The increased levels of these cytokines in similar co-culture conditions further corroborate the activation and functional response of CAR-T cells. The production of TNF-α and IFN-γ, both critical for effective immune responses against tumor cells, signifies the CAR-T cells' capability to recognize and respond to cancer cells displaying PD-L1.

Furthermore, the assay analyzed IL-10 and IL-6 levels, as seen in the lower-left graph of FIG. 13. The observed cytokine profiles provide additional layers of evidence supporting the functional activity of CAR-T cells in vitro. The differential cytokine secretion based on PD-L1 expression levels on target cells underscores the specificity and potential efficacy of the TT0002 CAR-T cells.

These results, as comprehensively illustrated in FIG. 13, thus validate the efficacy of TT0002 CAR-T cells in in vitro experiments. They demonstrate the cells' ability to produce a robust immune response upon encountering target cells with varying PD-L1 expressions, a critical indicator of their potential effectiveness in CAR-T cell therapy applications.

### 2.6 Enhanced Efficacy of TT0002 CAR-T Demonstrated in Xenograft Mouse Model

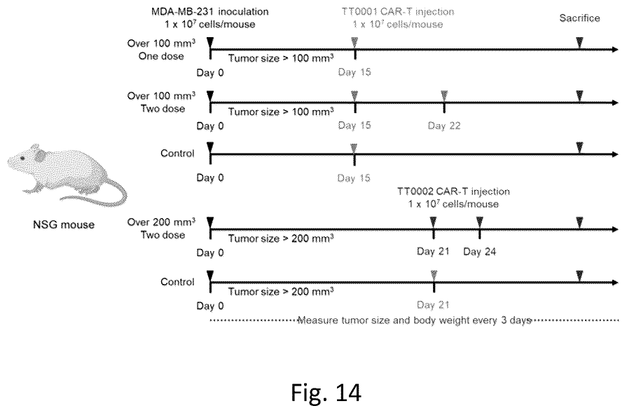

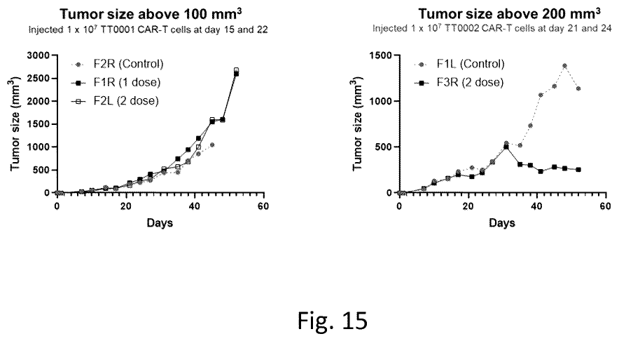

In the crucial phase of validating CAR-T cell therapies, in vivo efficacy studies provide indispensable insights. FIGS. 14-15 showcase the results of such a study, comparing the effectiveness of TT0001 and TT0002 CAR-T cells in a xenograft mouse model using NSG mice injected with MDA-MB-231 cells. This model is critical for assessing the real-world potential of these CAR-T cell therapies in targeting cancer.

In the experimental setup described in FIG. 14, TT0001 CAR-T cells were administered when tumor sizes exceeded 100 mm³, with a subsequent dose one week later. Conversely, the TT0002 CAR-T cells were introduced into a separate group of mice with more advanced tumor growth, exceeding 200 mm³, followed by an additional dose after three days. This approach aimed to compare the efficacy of both CAR-T cell types under variable tumor burdens.

FIG. 15 reveals a stark contrast in tumor suppression between the two CAR-T treatments. The TT0001 CAR-T cells, despite early intervention, failed to exhibit a significant impact on tumor size. On the other hand. TT0002 CAR-T cells demonstrated a pronounced ability to control tumor growth, maintaining tumor sizes at approximately 300 mm³ until day 52. This result not only highlights the superior efficacy of TT0002 CAR-T cells but also suggests their potential effectiveness in more advanced stages of tumor development.

The disparity in outcomes between TT0001 and TT0002 CAR-T treatments underscores the significance of CAR design and selection in developing effective cancer immunotherapies. The pronounced tumor suppression observed with TT0002 CAR-T cells in this xenograft model bolsters the case for their clinical application in cancer treatment, potentially offering a more effective therapeutic option for patients.

### 2.7 Demonstrated In Vivo Efficacy of TT0002 CAR-T in Xenograft Mouse Model

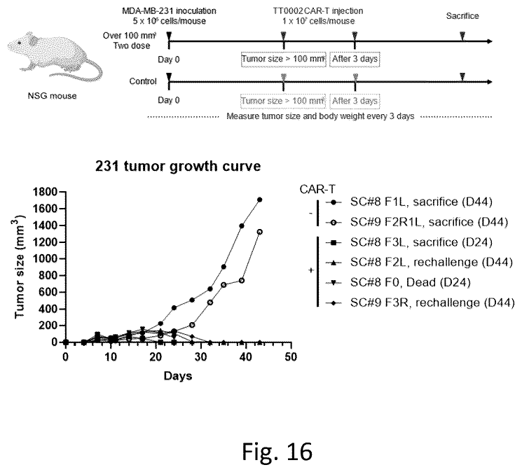

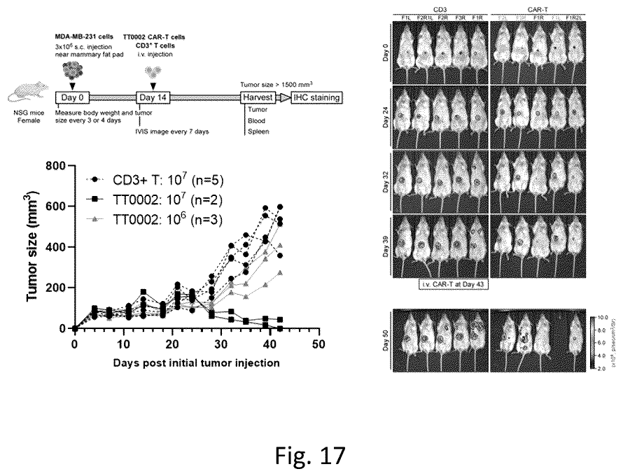

In a significant step towards validating the in vivo effectiveness of CAR-T therapies, our study focused on evaluating the impact of TT0002 CAR-T cells on tumor growth in a xenograft mouse model. The results, as referenced in FIGS. 16-17, offer compelling evidence of the potent tumor-suppressing capabilities of TT0002 CAR-T cells.

In the experiment detailed in FIG. 16, we observed a remarkable reduction in tumor growth following the administration of TT0002 CAR-T cells. The study involved reducing the initial tumor cell injection and then introducing TT0002 CAR-T cells upon the tumors reaching a measurable size. Notably, within approximately 30 days of TT0002 CAR-T cell treatment, a significant number of treated mice displayed an undetectable level of tumor growth, a stark contrast to the control group treated with PBS. This drastic reduction in tumor size highlights the potential of TT0002 CAR-T cells to effectively target and reduce tumor mass in a living organism.

Further reinforcing these findings, FIG. 17 details a dose-dependent study of TT0002 CAR-T's tumor suppression effects. The study demonstrates that higher doses of TT0002 CAR-T cells lead to a more pronounced reduction in tumor size, with some instances of complete tumor disappearance. This dose-dependent response provides valuable insights into the therapeutic window and efficacy of TT0002 CAR-T cells, suggesting their suitability for tailored cancer treatment regimens.

In conclusion, the in vivo experiments as substantiated by FIGS. 16-17, clearly demonstrate the dose-responsive tumor-suppressive efficacy of TT0002 CAR-T cells in a xenograft mouse model. These findings are pivotal in illustrating the practical application and potential of TT0002 CAR-T therapy in cancer treatment. The substantial reduction in tumor size, observed under various dosing conditions, provides essential data for the further development and optimization of TT0002 CAR-T cells as a targeted therapeutic approach in oncology. This evidence reinforces the promise of TT0002 CAR-T cells in contributing significantly to the advancement of effective cancer immunotherapies.

### 2.8 Longevity of TT0002 CAR-T Cells in Rechallenged Xenograft Mouse Model

The persistence and sustained efficacy of TT0002 CAR-T cells were evaluated in an extended in vivo study using a rechallenge xenograft mouse model. This study is pivotal in demonstrating the long-term therapeutic potential of CAR-T cells.

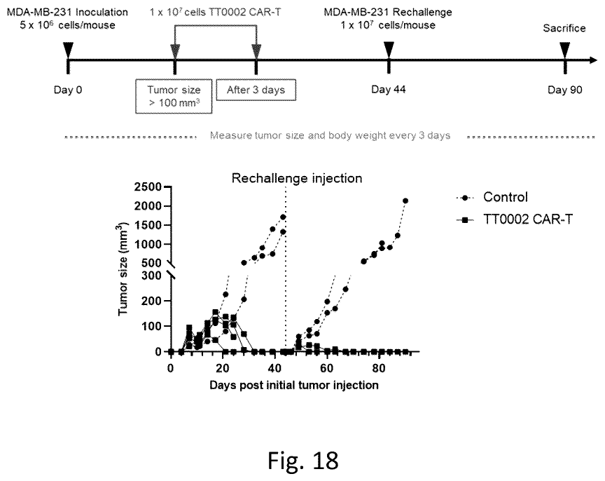

Initially, the study followed the progress of two mice previously treated with TT0002 CAR-T cells, which were later re-injected with MDA-MB-231 cells to assess tumor re-emergence. Over an extended observation period leading up to day 90, these mice exhibited no tumor growth, contrary to the control group. This outcome, as detailed in FIG. 18, highlights the sustained anti-tumor activity of TT0002 CAR-T cells, evidencing their efficacy over a substantial duration post-initial administration.

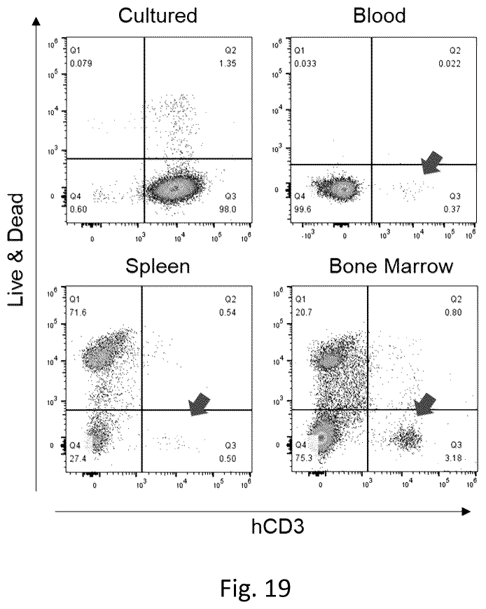

Subsequent analysis of blood, spleen, and bone marrow tissues from these mice provided insights into the distribution and persistence of CAR-T cells within the body. Flow cytometry results, presented in FIG. 19, showed a detectable presence of CAR-T cells, particularly in the bone marrow; suggesting a possible reservoir or site of prolonged CAR-T cell activity.

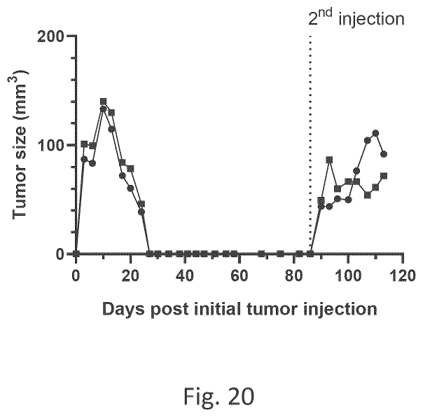

The study was further extended to day 86 to observe the long-term effects of CAR-T cell therapy. A significant reduction in tumor growth was observed, as shown in FIG. 20. This indicates that while the concentration of active CAR-T cells decreased over time, a residual population remained effective in suppressing tumor growth.

These findings collectively underscore the potential of TT0002 CAR-T cells for long-term cancer therapy.

### 2.9 Immunohistochemical Confirmation of TT0002 CAR-T Cell Penetration and Activation in Xenograft Tumors

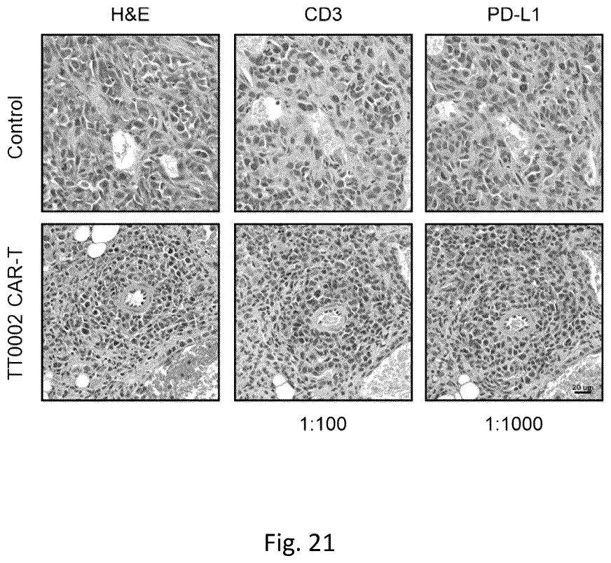

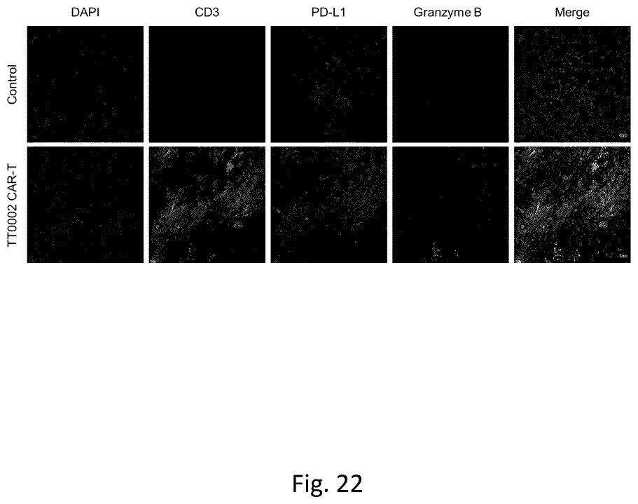

This section presents the results of immunohistochemical and immunofluorescence analyses performed to confirm the infiltration and activation of TT0002 CAR-T cells within xenograft tumor tissues, as visually supported by FIGS. 21-22.

FIG. 21 employed immunohistochemistry to detect the presence of CAR-T cells in the tumor tissues of mice treated with TT0002 CAR-T cells. Notably, in the control group where PBS was injected, a widespread expression of PD-L1 was observed in the tissue sections, but human CD3-expressing cells were notably absent. In stark contrast, the tissue sections from the TT0002 CAR-T treated group displayed a distinct presence of human CD3-expressing CAR-T cells, particularly around central blood vessels. This ring of CAR-T cells was surrounded by tissue exhibiting high PD-L1 expression, indicating a targeted infiltration of CAR-T cells into the tumor area.

FIG. 22 utilized immunofluorescence staining to further verify the presence and activity of CAR-T cells in the xenograft tumor tissues. The control group showed extensive PD-L1 expression without any significant presence of human CD3 or signs of T cell-mediated tumor cell killing. Conversely, in the TT0002 CAR-T treated group, human CD3 signals were evident and colocalized with PD-L1 signals. Additionally, a significant presence of Granzyme B signals was detected, indicative of the cytotoxic action of CAR-T cells on the target tumor cells.

These findings, illustrated in FIGS. 21-22, provide strong evidence of the successful infiltration and activation of TT0002 CAR-T cells in the tumor microenvironment. The ability of these cells to specifically target and exert cytotoxic effects on tumor cells while infiltrating the tumor tissue underscores the potential effectiveness of TT0002 CAR-T therapy in cancer treatment.

### 2.10 Biodistribution Analysis of CAR-T Cells

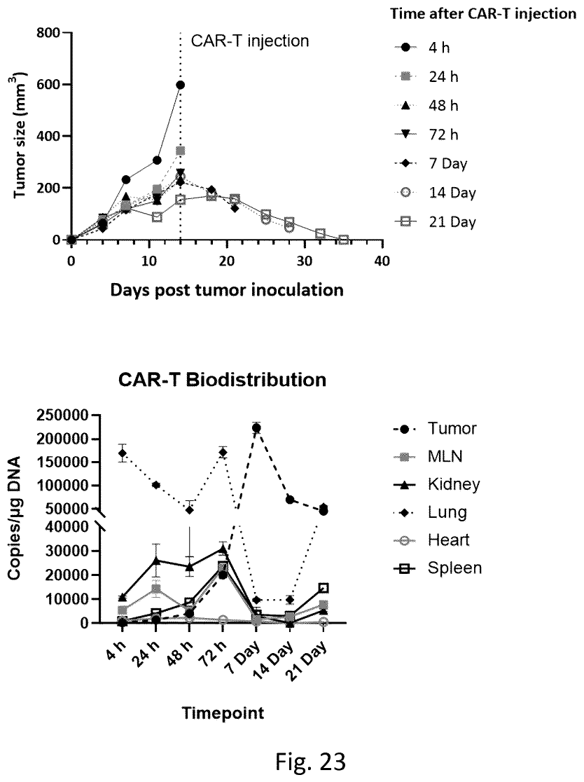

The results of the biodistribution study of TT0002 CAR-T cells, demonstrated in FIG. 23, provide critical insights into the distribution and persistence of these cells in an in vivo mouse model. This analysis is essential for understanding the migration and localization of CAR-T cells following their administration.

Post-injection of TT0002 CAR-T cells into mice with established tumors, the tumor growth curves (FIG. 23, left image) revealed a substantial reduction in tumor size after 35 days, aligning with 21 days post-injection. This reduction indicates the therapeutic impact of the CAR-T cells on tumor regression.

The CAR copy number in DNA samples, as shown in the right image of FIG. 23, was analyzed to determine the distribution of CAR-T cells within the mice. Notably, within the first 72 hours, a significant presence of CAR-T cells was observed in the lungs. However, by day seven post-injection, the primary localization of CAR-T cells shifted to the tumor sites. This migration pattern suggests an active homing mechanism, where CAR-T cells move towards and accumulate in tumor tissues, likely in response to tumor-specific antigens.

The biodistribution data presented in FIG. 23 underscores the targeted action of TT0002 CAR-T cells and their capacity for tumor-specific localization. The migration from initial lung distribution to tumor tissues highlights the cells' ability to actively seek and impact tumor sites, a critical factor in their therapeutic efficacy.

### 3. Conclusion

This study has presented comprehensive data and analyses underscoring the efficacy, specificity, and long-term potential of Chimeric Antigen Receptor T (CAR-T) cells in cancer treatment. The findings offer conclusive evidence supporting the innovative approach and effectiveness of the CAR-T therapy, particularly in targeting cancers expressing Programmed Death-Ligand 1 (PD-L1).

Efficacy in Target Recognition and Cytotoxicity: The immunohistochemistry assays have conclusively demonstrated the specific binding and targeted action of the CAR-T cells against PD-L1 expressing tumor cells as prepared herein. These results indicate a heightened sensitivity and specificity of the anti-PD-L1 CAR-T cells, making them a potent option for treating PD-L1 expressing cancers.

In Vitro and In Vivo Validation: The successful expression of CAR constructs in Jurkat cells and primary human CD3+ T cells and the subsequent demonstration of their cytotoxicity in various breast cancer cell lines validate the in vitro efficacy of the anti-PD-L1 CAR-T cells. Moreover, the in vivo studies in xenograft mouse models have further established the significant tumor suppression capabilities of the anti-PD-L1 CAR-T cells, even in advanced stages of tumor growth.

Longevity and Persistent Efficacy: The rechallenge experiments underscore the durability and sustained anti-tumor efficacy of the anti-PD-L1 CAR-T cells. The findings from these experiments are pivotal, indicating that the anti-PD-L1 CAR-T cells can offer long-term therapeutic benefits, a crucial factor for lasting cancer remission.

Biodistribution and Targeted Action: The biodistribution study demonstrates the targeted migration and localization of the anti-PD-L1CAR-T cells to tumor sites, highlighting their ability to actively home in on and exert therapeutic effects specifically at tumor locations.

In conclusion, the comprehensive data provided herein affirm the potential of the CAR-T therapy of the present invention as a highly effective, specific, and sustainable treatment modality in the realm of cancer immunotherapy. The specificity in targeting PD-L1 expressing cells, combined with the proven efficacy profile, positions the CAR-T therapy using the CAR-T cells targeting PD-L1 as described herein as a promising candidate for future clinical applications in oncology, offering new hope for patients battling cancers that express PD-L1.

### REFERENCES

1. Pardoll D M. The blockade of immune checkpoints in cancer immunotherapy. Nat Rev Cancer. 2012; 12(4):252-264. doi:10.1038/nrc3239
2. Twomey J D, Zhang B. Cancer Immunotherapy Update: FDA-Approved Checkpoint Inhibitors and Companion Diagnostics. AAPS J. 2021; 23 (2):39. doi:10.1208/s12248-021-00574-0
3. Li C-W, Lim S-O, Chung E M, et al. Eradication of Triple-Negative Breast Cancer Cells by Targeting Glycosylated PD-L1. Cancer Cell. 2018; 33 (2):187-201.e10. doi:https://doi.org/10.1016/j.ccell.2018.01.009
4. Benicky J, Sanda M, Brnakova Kennedy Z, et al. PD-L1 Glycosylation and Its Impact on Binding to Clinical Antibodies. J Proteome Res. 2021; 20 (1):485-497. doi:10.1021/acs.jproteome.0c00521
5. Li C-W, Lim S-O, Xia W, et al. Glycosylation and stabilization of programmed death ligand-1 suppresses T-cell activity. Nat Commun. 2016; 7(1):12632. doi:10.1038/ncomms12632
6. Neelapu S S, Tummala S, Kebriaei P, et al. Chimeric antigen receptor T-cell therapy—assessment and management of toxicities. Nat Rev Clin Oncol. 2018; 15(1):47-62. doi:10.1038/nrclinonc.2017.148
7. Shimabukuro-Vornhagen A, Böll B, Schellongowski P, et al. Critical care management of chimeric antigen receptor T-cell therapy recipients. CA Cancer J Clin. 2022; 72(1):78-93. doi:https://doi.org/10.3322/caac.21702
8. Liu H, Ma Y, Yang C, et al. Severe delayed pulmonary toxicity following PD-L1-specific CAR-T cell therapy for non-small cell lung cancer. Clin Transl Immunol. 2020; 9(10):e1154. doi:https://doi.org/10.1002/cti2.1154
9. Bajor M, Graczyk-Jarzynka A, Marhelava K, et al. PD-L1 CAR effector cells induce self-amplifying cytotoxic effects against target cells. J Immunother Cancer. 2022; 10(1):e002500. doi:10.1136/jitc-2021-002500
10. Yang Y, Li C-W, Chan L-C, et al. Exosomal PD-L1 harbors active defense function to suppress T cell killing of breast cancer cells and promote tumor growth. Cell Res. 2018; 28(8):862-864. doi:10.1038/s41422-018-0060-4
11. Huang H-C, Lai Y-J, Liao C-C, et al. Targeting conserved N-glycosylation blocks SARS-CoV-2 variant infection in vitro. eBioMedicine. 2021; 74. doi:10.1016/j.ebiom.2021.103712
12. Huang H-C, Liao C-C, Wang S-H, et al. Hyperglycosylated spike of SARS-COV-2 gamma variant induces breast cancer metastasis. Am J Cancer Res. 2021; 11(10):4994-5005.

1. A chimeric antigen receptor (CAR) comprising a PD-L1 binding domain which comprises
(a) a heavy chain variable region (V_{H}) which comprises a heavy chain complementary determining region (HC CDR1) of SEQ ID NO: 1, a heavy chain complementary determining region 2 (HC CDR2) of SEQ ID NO: 2, and a heavy chain complementary determining region 3 (HC CDR3) of SEQ ID NO: 3; and
(b) a light chain variable region (V_{L}) region which comprises a light chain complementary determining region 1 (LC CDR1) of SEQ ID NO: 4, a light chain complementary determining region (LC CDR2) of SEQ ID NO: 5, and a light chain complementary determining region 3 (LC CDR3) of SEQ ID NO: 6.

2. The CAR of claim 1, wherein the CAR further comprises a hinge domain, a transmembrane domain and an intracellular signaling domain.

3. The CAR of claim 1, wherein the CAR comprises the PD-L1 binding domain comprising, from N-terminus to C-terminus, the V_{H} and the V_{L} or the V_{L} and the V_{H}.

4. The CAR of claim 3, wherein the V_{H} and the V_{L} are linked by a linker.

5. The CAR of claim 2, wherein the intracellular signaling domain comprises at least one be selected from CD137 (4-1BB) signal domain, CD28 signal domain, CD27 signal domain, ICOS signal domain, CD3ζ signal domain and any combination thereof.

6. The CAR of claim 2, wherein the CAR comprises the amino acid sequence of SEQ ID NO: 14 or 15; or the CAR comprises the amino acid sequence of SEQ ID NO: 20 or 21.

7. A nucleic acid molecule comprising a nucleotide sequence encoding a CAR of claim 1.

8. The nucleic acid molecule of claim 6, which is a vector.

9. A cell comprising a nucleic acid molecule of claim 7.

10. A pharmaceutical composition comprising a cell of claim 9.

11. A method for treating a subject having a disease, disorder or condition associated with an elevated expression of a tumor antigen, comprising administering to the subject an effective amount of a cell genetically modified to express a CAR of claim 1.

12. The method of claim 11, wherein the tumor antigen is PD-L1.

13. The method of claim 11, wherein the subject is suffered from cancer.

14. The method of claim 13, wherein the cancer is selected from the group consisting of breast cancer, colon cancer, head and neck cancer, thyroid cancer, lung cancer, ovarian cancer, pancreatic cancer, gastric cancer, kidney cancer, glioblastoma and leukemia.

15-18. (canceled)
